(19) United States
(12) Patent Application Publication
(10) Pub. No.: US 2016/0068806 A1
(43) Pub. Date: Mar. 10, 2016

(54) COMPOSITIONS AND METHODS FOR PRECISE PATTERNING OF POSTERIOR NEUROECTODERM FROM HUMAN PLURIPOTENT STEM CELLS

(71) Applicant: WISCONSIN ALUMNI RESEARCH FOUNDATION, Madison, WI (US)

(72) Inventors: Randolph Scott Ashton, Madison, WI (US); Ethan Scott Lippmann, Madison, WI (US)

(21) Appl. No.: 14/496,796

(22) Filed: Sep. 25, 2014

Related U.S. Application Data
(60) Provisional application No. 61/882,221, filed on Sep. 25, 2013.
(60) Provisional application No. 61/970,689, filed on Mar. 26, 2014.

(51) Int. Cl. C12N 5/0797; C12N 5/0793

(52) U.S. Cl. CPC C12N 5/0623; C12N 5/0619; C12N 2506/03; C12N 2500/32; C12N 2500/38; C12N 2500/25; C12N 2501/155; C12N 2501/727; C12N 2501/415; C12N 2500/60; C12N 2501/119; C12N 2501/41; C12N 2501/19

(57) ABSTRACT

Described herein are methods, compositions, and kits for directed differentiation of human pluripotent stem cells into caudal lateral epiblasts, posterior neuroectoderm or posterior neuroepithelium, or motor neurons having specified HOX gene expression pattern mirroring a desired position along the rostral-caudal axis during hindbrain and spinal cord development. Also described are isolated populations of cells including caudal lateral epiblasts, posterior neuroectoderm, posterior neuroepithelium, or motor neurons having a HOX gene expression pattern specified to correspond to the HOX gene expression pattern associated with a desired rostral-caudal axis position.

### CROSS-REFERENCE TO RELATED APPLICATIONS

This application claims the benefit of U.S. provisional Application No. 61/882,221 filed on Sep. 25, 2013, and claims the benefit of U.S. provisional Application No. 61/970,689 filed on Mar. 26, 2014. Each of these applications is incorporated by reference herein in its entirety.

### STATEMENT REGARDING FEDERALLY SPONSORED REASEARCH OR DEVELOPMENT

Not applicable.

### BACKGROUND

Human pluripotent stem cells (hPSCs), including human embryonic stem cells (hESCs) and human induced pluripotent stem cells (hiPSCs), are powerful tools for studying human development and disease and may one day serve as a cell source for regenerative medicine. Significant advancements have been made in deriving neural stem cells from hPSCs and in their further differentiation to diverse neural lineages of the central nervous system (CNS) and peripheral nervous system (PNS). However, while researchers have made significant progress in differentiating human pluripotent stem cells (hPSCs) into neural cells patterned to specific regions of the anterior central nervous system (e.g. midbrain and forebrain), progress in effectively controlling hPSC specification to various segments of the hindbrain and spinal cord has been limited.

During development, rostrocaudal positional identity within the hindbrain and spinal cord is encoded by combinatorial expression of 39 HOX genes located within four paralogous genomic loci. HOX gene expression occurs in a spatially and temporally collinear manner. For example, motor neurons in the hindbrain primarily express rostral or 3′ Hox paralogs (e.g. Hox 1 to Hox4), and those in the spinal cord largely express caudal or 5′ Hox paralogs (e.g. Hox4 to Hox13). Specifically in the spinal cord, HOX expression demarcates rostrocaudal segments, with Hox4 to Hox8 being primarily expressed in the cervical/brachial spinal cord, Hox9 being expressed in the thoracic spinal cord, and Hox10-13 being expressed in the lumbar/sacral spinal cord. Moreover, HOX expression can also encode segment-specific neural phenotypes. For example, the HOX expression profile in motor neurons regulates their subtype specification, columnar and pool segmentation, and innervation targeting of muscle groups.

Accordingly, methods and compositions to derive neural cells (e.g., neurons and astrocytes) under defined conditions, with specific rostrocaudal hindbrain and spinal cord segmental identity (i.e. a detailed and predictable HOX expression profile) would be of great utility for disease modeling, regenerative therapy, and drug screening applications.

### BRIEF SUMMARY OF THE INVENTION

The invention relates generally to methods and compositions for directed differentiation of hPSCs into caudal lateral epiblasts, posterior neuroectoderm, or posterior neuroepithelium having specified HOX gene expression profiles mirroring various positions along the rostral (top/front of the hindbrain) hindbrain-caudal (bottom/“tail end” of the spinal cord) spinal cord axis. Also described are isolated populations of cells of caudal lateral epiblasts, posterior neuroectoderm, or posterior neuroepithelium having HOX gene expression profiles corresponding to the HOX gene expression pattern associated with a desired rostral (hindbrain)-caudal (lumbar spinal cord) axis position.

Accordingly, in a first aspect, described herein is a fully defined cell culture medium comprising water, salts, amino acids, vitamins, a carbon source, a buffering agent, selenium, insulin, an FGF (fibroblast growth factor), and an activator of β catenin pathway signaling, wherein the medium is substantially free of a TGFβ-signaling activator, and wherein the FGF is FGF2, FGF8a, FGF8b, FGF8f, FGF17, or FGF18.

In some embodiments, the fully defined medium further comprises GDF11 (growth differentiation factor 11). In some embodiments, the fully defined medium further comprises an inhibitor of BMP (bone morphogenetic protein) signaling (e.g., Dorsomorphin, noggin, DMH1, or LDN193189). In some embodiments the fully defined medium is substantially free of a TGFβ-signal (transforming growth factor beta signal) inhibitor. In some embodiments, the fully defined medium further comprises ascorbate. In some embodiments the fully defined medium further comprises a transferrin.

In some embodiments, the activator of β-catenin pathway signaling is a GSK3 (glycogen synthase kinase-3) kinase inhibitor. In some embodiments the GSK3 kinase inhibitor is CHIR99021.

In some embodiments the fully defined medium consists essentially of water, salts, amino acids, vitamins, a carbon source (e.g., glucose), a buffering agent, selenium, insulin, FGF8b, and an activator of β-catenin pathway signaling.

In a related aspect, described herein is a cell culture comprising Sox2₊/Brachyury⁺/PAX6⁻/Otx2⁻ human caudal lateral epiblasts and the above-described fully-defined medium.

In another related aspect, described herein is a kit comprising an FGF (e.g., FGF2, FGF8a, FGF8b, FGF17, or FGF18) and an activator of β-catenin pathway signaling. In some embodiments, the kit also includes a cell culture medium comprising water, salts, amino acids, vitamins, a carbon source, a buffering agent, selenium, and insulin. In some embodiments, the kit further includes a retinoid (e.g., retinoic acid). In some embodiments, the kit also includes GDF11. In some embodiments, the kit also includes an inhibitor of BMP signaling. In some embodiments, the kit further includes a retinoid, GDF11, and an inhibitor of BMP signaling.

In another related aspect, described herein is a method for generating caudal lateral epiblasts from human pluripotent stem cells, including the steps of: (i) culturing human pluripotent stem cells during a first culture period of about one to two days with a neural differentiation base medium to obtain a first cell population; (ii) culturing the first cell population for a second culture period of about one day to about four days in neural differentiation base medium supplemented with an FGF to obtain a second cell population, wherein the second cell population is Sox2⁺, Otx2⁺, Brachyury⁻ and Pax6⁻; and (iii) culturing the second cell population for a third culture period of about one day to about seven days in neural differentiation base medium supplemented with an FGF and an activator of β-catenin pathway signaling to obtain caudal lateral epiblasts that are Sox2⁺, Brachyury⁺, Pax6⁻ and Otx2⁻, wherein the neural differentiation base medium comprises water, salts, amino acids, vitamins, a carbon source, a buffering agent, selenium, and insulin, and wherein the FGF is FGF2, FGF8a, FGF8b, FGF8f, FGF17, or FGF18.

In some embodiments the activator of β-catenin pathway signaling is a GSK3 inhibitor. In some embodiments the third culture period is at least six days, and, after at least four days of the third culture period, the culture medium also includes GDF11 and an inhibitor of BMP signaling (e.g., dorsomorphin, DMH1, or LDN193189).

In another related aspect, provided herein is a method for generating posterior neuroectoderm or neuroepithelium from human pluripotent stem cells comprising culturing during a fourth culture period the Sox2⁺/Brachyury⁺/PAX6⁻/Otx2⁻ caudal lateral epiblasts obtained by the above-described method in neural differentiation base medium supplemented with a retinoid, wherein the fourth culture period lasts for about one to five days.

In some embodiments, the fourth culture period lasts for about four days. In some embodiments, the neural differentiation base medium supplemented with a retinoid further comprises an inhibitor of BMP signaling. In some embodiments where the method is used to obtain posterior neuroepithelium, the culturing during the fourth culture period is substantially free of an activator of β-catenin pathway signaling.

In some embodiments, the above-mentioned first cell population is provided as a cell monolayer.

In another related aspect, described herein is a method for generating human posterior neuroectoderm or neuroepithelium, comprising culturing a population of Sox2⁺/Brachyury⁺/PAX6⁻/Otx2⁻ human caudal lateral epiblasts that express at least one HOX gene in a neural differentiation base medium supplemented with a retinoid to obtain posterior neuroectoderm or neuroepithelium comprising a population of cells that are Sox2+/PAX6⁺/Brachyury⁻ and express at least one HOX gene, wherein the neural differentiation base medium comprises water, salts, amino acids, vitamins, a carbon source, a buffering agent, selenium, and insulin.

In some embodiments, the population of Sox2⁺/Brachyury⁺/PAX6⁻/Otx2⁻ human caudal lateral epiblasts are cultured in the retinoid-supplemented neural differentiation base medium for a period of about one to five days. In some embodiments, the population of Sox2⁺/Brachyury⁺/PAX6⁻/Otx2⁻ human caudal lateral epiblasts are cultured in the retinoid-supplemented neural differentiation base medium for a period of about four days. In some embodiments, the human caudal lateral epiblasts express Hoxd10. In some such embodiments, the supplemented neural differentiation base medium further comprises an inhibitor of BMP signaling (e.g., dorsomorphin).

In some embodiments, the human posterior caudal lateral epiblasts express Hoxc9. In some embodiments the human posterior caudal lateral epiblasts express Hoxc6. In some embodiments, the human posterior caudal lateral epiblasts express Hoxb4. In some embodiments, the human posterior caudal lateral epiblasts express Hoxa2.

In another related aspect, described herein is a method for generating a population of human motor neurons having a specified HOX gene expression profile, comprising culturing human posterior neuroectoderm or neuroepithelium having a specified HOX gene expression profile in a neural differentiation base medium supplemented with a retinoid and an activator of the Hedgehog signaling pathway to obtain the population of human motor neurons.

In some embodiments, the population of human motor neurons obtained has a higher level of a first specified HOX gene mRNA than a second specified HOX gene mRNA. In some such embodiments, the first HOX gene is Hoxa2 and the second HOX gene is Hoxb4. In some such embodiments, the first HOX gene is Hoxb4 and the second HOX gene is Hoxc6. In some such embodiments, the first HOX gene is Hoxc6 and the second HOX gene is Hoxc9. In some such embodiments, the first HOX gene is Hoxc9 and the second HOX gene is Hoxc6. In some such embodiments, the first HOX gene is Hoxc9 and the second HOX gene is Hoxd10. In some such embodiments, the first HOX gene is HoxD10 and the second HOX gene is Hoxc9.

In some embodiments, the culturing occurs for about 7 to 14 days.

In another related aspect, described herein is an isolated cell population consisting essentially of one of: (i) human caudal lateral epiblasts; (ii) human neuroectodermal cells; (iii) human neuroepithelial cells; and (iv) human motor neurons; wherein the isolated cell population has an mRNA expression profile characterized by a higher level of a first specified HOX gene mRNA than that of a second specified HOX gene mRNA. In some such embodiments, the first HOX gene is Hoxa2 and the second HOX gene is Hoxb4. In some such embodiments, the first HOX gene is Hoxc6 and the second HOX gene is Hoxc9. In some such embodiments, the first HOX gene is Hoxc9 and the second HOX gene is Hoxd10. In some such embodiments, the first HOX gene is Hoxd 10 and the second HOX gene is Hoxc9. In some such embodiments, the first HOX gene is Hoxb4 and the second HOX gene is Hoxc6.

In some embodiments, a plurality of the human caudal lateral epiblasts, neuroectodermal cells, or neuroepithelial cells are genetically modified. In some embodiments, the plurality of genetically modified human caudal lateral epiblasts, neuroectodermal cells, or neuroepithelial cells comprise an expression cassette or exogenous RNA encoding a fluorescent reporter protein, a growth factor, an extracellular matrix protein, or an antibody.

In another related aspect, provided herein is a cell culture comprising the above-mentioned isolated cell population and a neural differentiation base medium supplemented with an FGF and an activator of β-catenin pathway signaling, wherein the neural differentiation base medium comprises water, salts, amino acids, vitamins, a carbon source, a buffering agent, selenium, and insulin. In some embodiments, the FGF is FGF8b.

In another related aspect, provided herein is a cell culture comprising one of the above-mentioned isolated cell populations and a neural differentiation base medium supplemented with: (i) an FGF and an activator of β-catenin pathway signaling; (ii) a retinoid; or (iii); a retinoid and an inhibitor of BMP signaling, wherein the neural differentiation base medium comprises water, salts, amino acids, vitamins, a carbon source, a buffering agent, selenium, and insulin, and wherein the FGF is FGF2, FGF8a, FGF8b, FGF8f, FGF17, or FGF18. In some embodiments, (i) also includes GDF11.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

This application includes a sequence listing in computer readable form (a “txt” file) that is submitted herewith. This sequence listing is incorporated by reference herein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The present invention will be better understood and features, aspects and advantages other than those set forth above will become apparent when consideration is given to the following detailed description thereof. Such detailed description makes reference to the following drawings, wherein:

FIG. 1 shows (A) a schematic representation of the four HOX genes clusters in the human genome, and (B) mRNA expression domains of select HOX genes throughout the human hindbrain and spinal cord segments, as estimated from murine and chick models. HoxC6, C9, and D10 expression broadly delineate specific spinal cord regions.

FIG. 2. (A) A schematic of experiments to generate Pax6⁻ caudal lateral epiblasts with flow cytometry analysis of Sox2 and Brachyury expression; ±S.D. calculated from two biological replicates. (B) A schematic of caudal neural tube formation. Wnts and FGF8 maintain the caudal lateral epiblast-containing stem zone while flanked by presomitic mesoderm (PSM). Wnts/FGF8 decline in the transition zone upon node/primitive streak regression, and newly formed somites secrete retinoic acid (RA), leading to neuroectoderm and then neuroepithelium formation within the neural tube. (C) HOX gene activation in response to FGF8b (100 ng/ml) and CHIR 99021 (3 μM) over four days of treatment.

FIG. 3. (A) (upper panel) A schematic overview of differentiation of hESCs to caudal lateral epiblasts followed by various treatments to determine their effect on HOX gene expression/progression; and (lower panel) a photograph of an agarose gel with RT-PCR reactions for HOX gene expression after removal of FGF8b (F, 200 ng/ml) and/or CHIR 99021 (C, 4 μM) while adding RA (3 μM) or no addition. (B) Cells from lane 5 in panel A analyzed for human neuroepithelial markers. (C) Cells maintained as caudal lateral epiblasts for two days (2D FGF8b/CHIR 99021) or five days (5D Fgf8b/CHIR 99021), then differentiated with RA and probed for HoxB4 expression.

FIG. 4 shows that Wnt/β-catenin and FGF signaling coordinate HOX propagation during hPSC differentiation. Cell lines were passaged for differentiation on day −1 for all experiments (see Example 4 for details). (A) Timeline of H9 hESC differentiation corresponding to panels (B) and (C). RA, 1 μM; CHIR 99021, 3 μM; FGF8b, 100 ng/ml. (B) Gene expression analysis by RT-PCR. Control sample is E6 medium alone. (C) qPCR analysis at day 5 of differentiation normalized to the morphogen treatment that yielded maximum expression. Statistical significance was calculated using the Student's unpaired t-test relative to the sample with maximum expression (*, p<0.005; **, p<0.001). (D) qPCR analysis of gene expression at day 2 using the H9 ishcat2 line in the presence or absence of doxycycline (2 μg/ml) added 3 days prior to morphogen treatment. Data are normalized to doxycycline-free samples for each respective morphogen and statistical significance was calculated using the Student's unpaired t-test (*, p<0.01). (E) qPCR analysis of gene expression at day 5 using the H9 ishcat2 line in the presence or absence of doxycycline. Statistical significance was calculated using the Student's unpaired t-test (*, p<0.005). (F) Inhibition of RA signaling by HX531 does not disrupt HOX progression. [HX531]=1 μM. Timeline shown on left panel. RT-PCR data (right panel) indicates that CHIR/FGF8b effects are not indirectly mediated by endogenous RA signaling.

FIG. 5 shows colinear HOX expression in hPSC-derived neuromesodermal progenitors (AKA Sox2⁺/Brachyury⁻/Pax6⁻/Otx2⁻ caudal lateral epiblasts). (a) Neuromesodermal, a.k.a., identity was evaluated when FGF signaling was initiated concurrently or 24 h prior to Wnt/β-catenin signaling. (b) Quantification of neuromesodermal identity. Flow cytometry data are presented as mean±standard deviation calculated from two biological replicates (grey histogram, IgG control; red histogram, label of interest). Scale bars in immunofluorescence images indicate 100 μm and adjacent horizontal images represent the same field. (c) Optimized morphogen treatment schedule evaluated in the presence and absence of growth differentiation factor 11 (GDF11). The addition of CHIR 99021 is denoted as t=0 h in panels D-F. (d) Purity of neuromesodermal progenitors in the presence or absence of GDF11. Expression was quantified by flow cytometry and presented as mean±standard deviation calculated from at least two biological replicates. (e) Quantification of HoxC10 and HoxD10 expression in the neuromesodermal state by qPCR in the presence or absence of GDF11. Each gene is normalized to its maximum expression. Error bars are standard deviation calculated from duplicate reactions. (F) Quantification of colinear HOX expression in the neuromesodermal state by qPCR. GDF11 was included according to panel C. Each gene is normalized to its maximum expression. Error bars are standard deviation calculated from duplicate reactions. (G) Time series expression for all recorded HOX genes.

FIG. 6 shows that multiple FGF isoforms can be used for differentiation of hPSCs to neuromesodermal cells and activation of Hox1-5 paralogs. (a) Timeline of differentiation. (b)-(g) Use of FGF2 (b), FGF8a (c), FGF8b (d), FGF8f (e), FGF17 (f), or FGF18 (g) in the protocol outlined in (a) yields cell populations with nearly uniform expression of Sox2 and T (Brachyury). (H) Agarose gel analysis for HOX gene expression after two days of FGF/CHR treatment in the protocol outlined in (a). Use of FGF8a (first lane), FGF8b (second lane), FGF8f (third lane), FGF2 (fourth lane), FGF17 (fifth lane) or FGF18 (fifth lane) all yield cell populations having activated Hox1-5 paralogs.

FIG. 7 shows that dorsomorphin is required during GDF11 treatment to achieve Pax6 expression after RA addition. (A) Timeline of differentiation. (B) qPCR assessment of lumbar/sacral gene expression at day 12. A minimum concentration of GDF11 is required to activate HoxD10 and HoxA11. Each gene is normalized to the maximum GDF11 dose. Error bars are standard deviation calculated from duplicate reactions. (C) Expression of Pax6 in response to increased GDF11 concentrations. Data were collected by flow cytometry and represented as mean±standard deviation calculated from two biological replicates. Sox2 was uniformly expressed regardless of GDF 11 treatment (data not shown). (D) Flow cytometry histograms demonstrating that the addition of dorsomorphin (DM) is sufficient to recover Pax6 expression (83±4%). Grey histograms, IgG control; Red histograms, Pax6. Data are representative of two biological replicates. (E) Dorsomorphin does not affect the acquisition of HoxD10. Percentages were quantified as HoxD10 labeling relative to DAPI-stained nuclei (>2500 total cells counted per sample). Scale bars, 100 μm

FIG. 8 (A) shows that retinoic acid (RA) specifies neural fate and defined positional identity. Assessment of neuroectoderm or mesoderm fate transition by flow cytometry. Grey histogram, IgG control; red histogram, antigen of interest. Data are presented as mean±standard deviation calculated from two biological replicates. (B) For experiments in panels C-F, neuromesodermal propagation was halted by removing FGF8b/CHIR 99021/GDF11 and adding RA. Assays were conducted after 4 days of RA treatment. (C) HOX profiles generated after RA treatment. Each gene is normalized to its maximum expression. Error bars are standard deviation calculated from duplicate reactions. (D) HoxB4 and HoxD10 expression in neuroepithelial cultures after RA treatment. Scale bars, 100 μm. (E) Purity of Sox2, Pax6, HoxB4, and HoxD10 after 4 days of RA treatment. Sox2, Pax6, and HoxB4 were quantified using flow cytometry and HoxD10 was quantified by manual counting of dissociated cells (N=4,5087 cells). Error bars are standard deviation calculated from at least two biological replicates. Dorsomorphin was required to rescue Pax6 expression after GDF11 treatment due to its dorsalizing capabilities but did not affect Sox2 or HoxD10 expression. (F) Motor neuron differentiation at cervical, thoracic, and lumbar spinal cord depths, where the motor neuron precursors show region-specific patterns of FoxP1, Isl1, and Hb9 co-expression. (G) Neuronal maturation from cervical, thoracic, and lumbar patterned NSCs, as assessed by immunocytochemistry (top panel; DAPI (blue) is overlaid in all images; Scale bars, 20 μm) and RT-PCR (50 cycles; bottom panel). RT-PCR demonstrates maintenance of positional identity.

FIG. 9 (A) provides additional data showing that RA induces a neuroectodermal fate and halts colinear HOX activation, using the neural differentiation scheme shown in FIG. 8B and representative hindbrain cultures assessed by qPCR. (B) Representative hindbrain cultures assessed by immunocytochemistry. (C) Representative hindbrain cultures assessed by flow cytometry. All data are presented as mean±S.D. and qPCR data are normalized to the time point of maximum expression for each gene. For the hindbrain cultures, HoxB1 and HoxB4 were quantified by immunocytochemistry relative to DAPI+nuclei, while Sox2 and Pax6 were quantified by flow cytometry. Scale bars, 100 μm.

FIG. 10 shows mass spectrometry comparison of Hox profiles in cervical, thoracic, and lumbar NSC cultures. Cervical differentiation: 1 d FGF8b, 2 d FGF8b/CHIR, 4 d RA; thoracic differentiation: 1d FGF8b, 6 d FGF8b/CHIR, 4 d RA; lumbar differentiation: 1 d FGF8b, 4 d FGF8b/CHIR, 2 d FGF8b/CHIR/GDF11, 4 d RA. Data are presented as mean±S.D. and statistical significance was calculated using the Student's unpaired t-test. *, p<0.01; **, p<0.002; ***, p<0.0001.

FIG. 11 shows neuromesodermal differentiation from IMR90-4 iPSCs. (A) Timeline of neuromesodermal differentiation. (B) Neuromesodermal identity assessed during differentiation in 2 or 3 μM CHIR 99021. Grey histograms, IgG control. Red histograms, label of interest. Data are presented as mean±standard deviation calculated from two biological replicates. Whereas 2 μM CHIR 99021 maintained the neuromesodermal state, 3 μM CHIR 99021 resulted in a mesodermal shift exemplified by a reduction in Sox2 expression. (C) Colinear HOX activation observed by qPCR during neuromesodermal differentiation. Each gene is normalized to its maximum expression. Error bars are standard deviation calculated from duplicate reactions.

FIG. 12 is a schematic overview of rostro-caudal specification of HOX identity in hPSC-derived neuromesodermal cells (caudal lateral epiblasts). Exposure of hPSCs to FGF and activators of the β-catenin signaling pathway for varying amounts of time drives expression of Hox family genes in a cervical to lumbar gradient. Shorter exposure periods drive expression of Hox genes expressed in cervical regions (e.g., Hoxa4), with longer exposure times driving expression of thoracic (e.g., C6) and lumbar (e.g., D10) HOX expression. Treatment with retinoids (e.g., retinoic acid-RA) arrests progression of the HOX spatiotemporal expression program and induces formation of neuroectoderm with a fixed HOX expression identity.

FIG. 13 shows the effects of various inhibitors on neuromesodermal differentiation. (A) Timeline of neuromesodermal differentiation and inhibitor addition. (B) Neuromesodermal identity assessed by Sox2 and Brachyury (T) expression after two days. Grey histograms, IgG control. Red histograms, label of interest. LY, LY-294002; U0126, U0126; Gf, GF109203X. All cultures were 99% Sox2⁺ and 70-85% Brachyury⁺, indicating no shift to an exclusive mesoderm identity.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

### I. In General

The present invention relates to the inventors' unexpected finding that under fully defined conditions, differentiation of hPSCs by application of certain FGF isoforms in combination with β-catenin signaling activation (e.g., via activation of Wnt signaling), followed by timed application of a retinoic acid receptor agonist, allows precise positional patterning of the resulting posterior neuroectoderm and neuroepithelium along the hindbrain-spinal axis, as reflected by HOX family gene expression. Accordingly, the methods and compositions described herein permit the generation of caudal lateral epiblasts, posterior neuroectodermal, and posterior neuroepithelial cell populations corresponding to specified positions along the rostral-caudal axis, based on the HOX gene expression patterns of such populations. Such a cohort of distinct cell populations, which were not obtainable from human pluripotent stem cells by previous methods, have differing functional properties, e.g., their potential to differentiate into various types of neurons or astrocytes found at distinct and different positions along the hindbrain-spinal cord.

In the Examples below, we utilized a fully defined, monolayer culture system that efficiently differentiated hPSCs into pure neuroectoderm. We demonstrated that while both RA and Wnt/β-catenin can activate HOX1-5 expression, only Wnt/β-catenin signaling can reduce Otx2 expression, which is a hallmark of posterior neural fate in vitro. Moreover, co-activation of Wnt/β-catenin and FGF signaling induced HOX1-9 expression in a temporal manner. Activation of FGF signaling upstream of Wnt/β-catenin signaling was necessary to establish a highly pure Sox2+/Brachyury+ neuromesodermal progenitors reminiscent of the axial stem cell population found in vivo, and these neuromesodermal progenitors demonstrated colinear HOX activation across each of the 4 HOX loci. While FGF and Wnt/β-catenin were sufficient for HOX propagation from the hindbrain thru the thoracic spinal cord, addition of GDF11 was necessary to activate lumbosacral paralogs.

At any point during this process, the transition to RA was sufficient to differentiate the neuromesodermal progenitors to definitive Pax6+/Sox2+ neuroectoderm. Moreover, RA acted as the ‘stop’ signal for HOX progression, thus preventing the acquisition of more caudal HOX paralogs that would otherwise become expressed under continuous FGF and Wnt/β-catenin signaling. Definitive Hox domains were established after RA addition, depending on which HOX factors were activated before RA treatment, including domains characteristic of rhombomere segments in the hindbrain and distinct cervical, thoracic, and lumbar/sacral regions in the spinal cord. Differentiation of cervical, thoracic, and lumbar/sacral neuroectoderm to motor neurons yielded phenotypic differences characteristic of the expected motor columns at each rostrocaudal location, and these populations could be terminally differentiated while still retaining their HOX profile. In general, the methods presented herein provide access to neuroectoderm and its resultant progeny having the characteristics of any location in the hindbrain and spinal cord.

A number of different FGF isoforms can be used in the disclosed compositions and methods. Non-limiting examples of suitable FGFs include FGF2, FGF8a, FGF8b, FGF8f, FGF17, and FGF18.

A number of retinoic acid receptor agonists can be used in the disclosed compositions and methods to stop HOX gene progression of differentiating hPSCs when the HOX gene expression pattern corresponds to that of a specific target location on the hindbrain-spinal cord axis. An exemplary class of suitable retinoic acid receptor agonists are the retinoids and retinoid analogs, which include without limitation All-Trans Retinoic Acid (ATRA), Retinol Acetate, EC23 (4-[2-(5,6,7,8-Tetrahydro-5,5,8,8-te-tramethyl-2-naphthalenyl)ethynyl)-benzoic acid; CAS No: 104561-41-3), BMS453 (4-[(1E)-2-(5,6-Dihydro-5,5-dimethyl-8-phenyl-2-naphthalenyl)ethenyl]-benzoic acid; CAS No: 166977-43-10), Fenretinide (N-(4-Hydroxyphenyl)retinamide; CAS No: 65646-68-6), AM580 (4-[(5,6,7,8-Tetrahydro-5,5,8,8-tetramethyl-2-naphthalenyl)carboxamido]benzoic acid; CAS No: 102121-60-8), Tazarotene (6-[2-(3,4-Dihydro-4,4-dimethyl-2H-1-benzothiopyran-6-yl)ethynyl]-3-pyridinecarboxylic acid ethyl ester; CAS No: 118292-40-3), and TTNPB (4-[(E)-2-(5,6,7,8-Tetrahydro-5,5,8,8-tetramethyl-2-naphthalenyl)-1-propenyl]benzoic acid; CAS No: 71441-28-6). Other exemplary retinoic receptor agonists that could be used include AC261066 (4-[4-(2-Butoxyethoxy-)-5-methyl-2-thiazolyl]-2-fluorobenzoic acid; CAS No: 870773-76-5), AC55649 (4′-Octyl-[1,1′-biphenyl]-4-carboxylic acid; CAS No: 59662-49-6), Adapalene (6-(4-Methoxy-3-tricyclo[3.3.1.13,7]dec-1-ylphenyl)-2-naphthalenecarboxylic acid; CAS No: 106685-40-9), AM80 (4-[[(5,6,7,8-Tetrahydro-5,5,8,8-tetramethyl-2-naphthalenyl)amino]carbonyl]benzoic acid; CAS No: 94497-51-5), BMS753 (4-[[(2,3-Dihydro-1,1,3,3-tetramethyl-2-oxo-1H-inden-5-yl)carbonyl]amino]benzoic acid; CAS No: 215307-86-1), BMS961 (3-Fluoro-4-[[2-hydroxy-2-(5,5,8,8-tetramethyl-5,6,7,8,-tetrahydro-2-naphthalenyl)acetyl]amino]-benzoic acid; CAS No: 185629-22-5), CD1530 (4-(6-Hydroxy-7-tricyclo[3.3.1.13,7]dec-1-yl-2-naphthalenyl)benzoic acid; CAS No: 107430-66-0), CD2314 (5-(5,6,7,8-Tetrahydro-5,5,8,8-tetramethyl-2-anthracenyl)-3-thiophenecarboxylic acid; CAS No: 170355-37-0), CD437 (6-(4-Hydroxy-3-tricyclo[3.3.1.13,7]dec-1-ylphenyl)-2-naphthalenecarboxylic acid; CAS No: 125316-60-1), and Ch55 (4-[(1E)-3-[3,5-bis(1,1-Dimethylethyl)phenyl]-3-oxo-1-propenyl]benzoic acid; CAS No: 110368-33-7).

Furthermore, certain retinoic acid receptor agonists that can be used in the disclosed compositions and methods may halt HOX gene progression without promoting transition of the cells to neuroectoderm, thus offering additional control over the developmental process.

The disclosed compositions and methods can facilitate the production of neural cells having a specific rostrocaudal hindbrain and spinal cord segmental identity, as manifested by a detailed and predictable HOX gene expression profile. Such cells would be of great utility for disease modeling, regenerative therapy, and drug screening applications.

The scalable derivation of human tissues with region-specific and discrete HOX profiles enabled by the disclosed methodology could be invaluable to elucidating the molecular mechanisms that govern cross-repressive Hox interactions and Hox regulation of neural cell fate. It is sometimes difficult to observe the behavior of intermediate cell populations that are not subjected to competing effects from other cell types in vivo (e.g., neuroectoderm without exogenous influence from differentiating progenitors and flanking mesoderm). Because the disclosed systems and methods can capture “snapshots” of developing cell phenotypes, they may have great utility for dynamic studies of hindbrain and spinal cord development that are intractable in humans, especially in combination with advancements in the genetic manipulation of cultured cells.

Specifically regarding disease modeling and regenerative therapies, HOX expression patterns are crucial determinants of cellular phenotype, organization, and neural circuit integration in the developing hindbrain and spinal cord. The disclosed systems and methods permit the generation of highly pure NSC cultures possessing a defined positional identity within the hindbrain or spinal cord that can be predicted a priori in a fully defined and scalable way. Recent studies of cell replacement therapy in the anterior CNS using hPSC-derived progenitors have demonstrated that the derived cells must possess a spatial identity and phenotype that mimics the endogenous tissue in order to effectively engraft and correct a neural deficit (see, e.g., Kriks, S. et al., Nature 480, 547-551 (2011); Ma, L. et al., Cell Stem Cell 10, 455-464 (2012), and Liu, Y. et al., Nat Biotechnol 31, 440-447 (2013).47-49). Accordingly, the methods presented herein could serve as the basis for generating a spectrum of posterior neural progeny that could aid regenerative therapy efforts.

Furthermore, motor neurons exhibit differential susceptibility to several neurodegenerative disorders, including amyotrophic lateral sclerosis (ALS) and spinal muscular atrophy (SMA), depending on their rostrocaudal identity (see Kanning, K. C., Kaplan, A. & Henderson, C. E., Annu Rev Neurosci 33, 409-440 (2010)). Because studies between iPSC-derived motor neurons with and without disease-causing mutations continue to identify relevant pathways in disease progression (see, e.g., Kiskinis, E. et al., Cell Stem Cell 14, 781-795 (2014); Chen, H. et al., Cell Stem Cell 14, 796-809 (2014)), analysis of the molecular differences between motor neurons with analysis of the molecular differences between motor neurons with defined rostrocaudal and columnar identities in the context of these disease models could yield further insights into the mechanisms of neurodegeneration.

In addition, hPSC-derived neurons having a specific rostrocaudal hindbrain and spinal cord segmental identity can be used in toxicity and drug screening. For example, such cells could be used to identify toxins that give rise to certain neural diseases associated with specific positions of the hindbrain and spinal cord, as well as for studying various manifestations of developmental neuritoxicity.

In sum, the ability to differentiate hPSCs to caudal neural tissue and to precisely control the positional identity of such tissue would enable significant advances in these fields.

### II. Definitions

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

In describing the embodiments and claiming the invention, the following terminology will be used in accordance with the definitions set out below.

As used herein, the term human “pluripotent stem cell” (hPSC) means a cell capable of continued self-renewal and capable, under appropriate conditions, of differentiating into cells of all three germ layers. Examples of hPSCs include human embryonic stem cells (hESCs) and human induced pluripotent stem cells (hiPSCs). As used herein, “iPS cells” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells.

As used herein, “about” means within 10% of a stated concentration range or within 10% of a stated time frame.

“Activator of β-catenin pathway signaling,” as used herein, means an agent that directly or indirectly increases β-catenin signaling in a cell. Examples of such agents include, but are not limited to, any of activators of Wnt pathway signaling (e.g., Wnt3a), GSK3 kinase inhibitors, and agents for inducing β-catenin overexpression.(e.g., overexpression vectors).

The term “defined culture medium” or “defined medium,” as used herein, means that the chemical structure and quantity of each individual ingredient in the medium is definitively known and independently controlled.

As used herein, “a medium consisting essentially of” means a medium that contains the specified ingredients and that may contain additional ingredients that do not materially affect its basic characteristics.

As used herein, “effective amount” means an amount of an agent sufficient to evoke a specified cellular effect according to the present invention.

“Hox media” refer to a group of media that modulate expression of Hox genes in hPSC-derived caudal lateral epiblasts.

“Hox-Start medium,” as used herein, refers to a medium that initiates progressive Hox gene family expression with rostral Hox genes (e.g., Hoxa1) being expressed initially followed by caudal Hox gene expression (e.g., expression of Hoxd10).

“Hox-Stop medium,” as used herein, refers to a medium that arrests the progression of Hox gene family expression and drives conversion of PAX6⁻ caudal lateral epiblasts to PAX6+ posterior neuroectoderm and neuroepithelium.

“Neural differentiation base medium,” as used herein, refers to a medium capable of promoting and supporting differentiation of human pluripotent stem cells towards a neural lineage, e.g., towards neuroectoderm and neuroepithelium. A neural differentiation base medium can include, but is not limited to E6 medium, as described herein and in U.S. Patent Publication No. 2014/0134732.

The terms “purified” or “enriched” cell populations are used interchangeably herein, and refer to cell populations, ex vivo, that contain a higher proportion of a specified cell type or cells having a specified characteristic than are found in vivo (e.g., in a tissue).

As used herein, an “mRNA expression profile” when referring to a cell population means the level of various RNA in the cell population as a whole, i.e., in an RNA sample extracted from the entire cell population, even though, there may be variation and deviation of mRNA expression profiles in individual cells or subpopulations from the cell population as a whole. For example if the mRNA expression profile of an isolated neuroepithelial cell population indicates that Hoxd10 mRNA is at a higher relative level than Hoxc9, this does not indicate that every individual cell in the population necessarily expresses Hoxd10 at a higher level than Hoxc9.

“Supplemented,” as used herein, refers to a composition, e.g., a medium comprising a supplemented component (e.g., an FGF). For example a medium “further supplemented” with an FGF, refers to the medium comprising FGF, and not to the act of introducing the FGF to the medium.

As used herein, “viability” means the state of being viable. Pluripotent cells that are viable attach to the cell plate surface and do not stain with the dye propidium iodide absent membrane disruption. Short term viability relates to the first 24 hours after plating the cells in culture. Typically, the cells do not proliferate in that time.

As used herein, “pluripotency” means a cell's ability to differentiate into cells of all three germ layers.

### III. Compositions

Cell Culture Media for Differentiation of hPSCs into Caudal Lateral Epiblasts

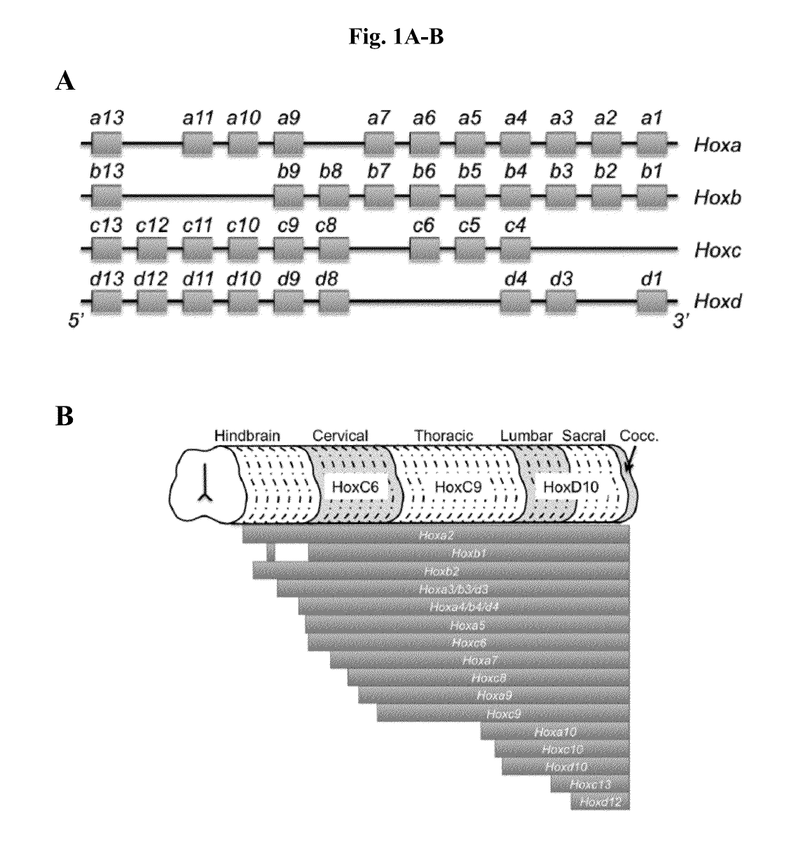

During development, the neural tube develops from head to tail (i.e. rostral-to-caudal direction). Similarly, HOX genes are expressed in a rostrocaudal collinear fashion, where, e.g., Hoxa 1 is expressed earliest and in anterior hindbrain positions, HoxB4 is expressed later and in more caudal hindbrain and cervical and brachial spinal cord tissues, and HoxD10 is expressed even later and in lumbar and potentially sacral spinal cord tissues (see also FIG. 1). Described herein are media specifically formulated to support differentiation of hPSCs into caudal lateral epiblasts, posterior neuroectoderm, or neuroepithelium patterned to various positional fates along the rostrocaudal axis, i.e., brainstem at the rostral end to lumbar/sacral spinal cord on the caudal end, which is reflected by Hox gene expression patterns as mentioned above. For ease of reference, such media are referred collectively herein as “Hox Media.”

TABLE 1
Exemplary Neural Differentiation Base Formulation
Components for HOX media

| | Formulation | | | |
|---|---|---|---|---|
| | 1 | 2 | 3 | 4 |
| DMEM/F12* | + | + | + | + |
| Selenium | + | + | + | + |
| Insulin | + | + | + | + |
| L-Ascorbic Acid | − | + | − | + |
| (Ascorbate) | | | | |
| Transferrin | − | − | + | + |

*or similar basal medium buffered to physiological pH (about 7.4) with bicarbonate or another suitable buffer such as HEPES. Osmolarity of the medium was adjusted to about 340 mOsm.

The final concentrations of the above listed basal medium components in exemplary Hox media are listed in Table 2:

TABLE 2
Concentrations of Neural Differentiation Base Formulation
Components Found in Exemplary HOX Media

| | Final | |
|---|---|---|
| Component | Concentration | |
| Sodium Selenite | 14 | μg/L |
| Insulin | 19.4 | mg/L |
| L-Ascorbic Acid | 64 | mg/L |
| Transferrin | 10.7 | mg/L |

The various Hox media described herein can be prepared starting from separate individual ingredients. Alternatively, one of skill in the art appreciates the efficiency of using a basal medium such as DMEM/F12 as starting material to prepare the disclosed Hox media. The term “basal medium” as used herein means a minimal medium that contains essentially water, salts, amino acids, vitamins, a carbon source, and a buffering agent. Such basal medium components are known in the art , e.g., a carbon source can include glucose, fructose, maltose, galactose. Other components that do not change the basic characteristic of the medium but are otherwise desirable can also be included, such as the pH indicator phenol red. For example, Dulbecco's Modified Eagle Medium: Nutrient Mixture F-12 (DMEM/F12) is a basal medium commonly used to make suitable growth media for mammalian cell culture. A complete list of ingredients of DMEM/F12 is set forth in Table 3.

TABLE 3
DMEM: F-12 Medium Formulation (ATCC Catalog No. 30-2006).

| Inorganic Salts (g/liter) | Amino Acids (g/liter) | Vitamins (g/liter) | Other (g/liter) |
|---|---|---|---|
| CaCl₂ (anhydrous) | L-Alanine 0.00445 | D-Biotin 0.00000365 | D-Glucose 3.15100 |
| 0.11665 | L-Arginine•HCl 0.14750 | Choline Chloride 0.00898 | HEPES 3.57480 |
| CuSO₄ (anhydrous) | L-Asparagine•H₂O | Folic Acid 0.00265 | Hypoxanthine 0.00239 |
| 0.0000008 | 0.00750 | myo-Inositol 0.01261 | Linoleic Acid 0.000044 |
| Fe(NO₃)3•9H₂O 0.00005 | L-Aspartic Acid 0.00665 | Niacinamide 0.00202 | Phenol Red, Sodium Salt |
| FeSO₄•7H₂O 0.000417 | L-Cystine•HCl•H₂O | D-Pantothenic Acid | 0.00810 |
| MgSO₄ (anhydrous) | 0.01756 | 0.00224 | Putrescine•2HCl 0.00008 |
| 0.08495 | L-Cystine•2HCl 0.03129 | Pyridoxine•HCl 0.00203 | Pyruvic Acid•Na 0.05500 |
| KCl 0.3118 | L-Glutamic Acid 0.00735 | Riboflavin 0.00022 | DL-Thioctic Acid |
| NaHCO₃ 1.20000 | L-Glutamine 0.36510 | Thiamine•HCl 0.00217 | 0.000105 |
| NaCl 7.00000 | Glycine 0.01875 | Vitamin B-12 0.00068 | Thymidine 0.000365 |
| Na₂HPO₄ (anhydrous) | L-Histidine•HCl•H₂O | | |
| 0.07100 | 0.03148 | | |
| NaH₂PO₄•H₂O 0.06250 | L-Isoleucine 0.05437 | | |
| ZnSO₄•7H₂O 0.000432 | L-Leucine 0.05895 | | |
| | L-Lysine•HCl 0.09135 | | |
| | L-Methionine 0.01724 | | |
| | L-Phenylalanine 0.03548 | | |
| | L-Proline 0.01725 | | |
| | L-Serine 0.02625 | | |
| | L-Threonine 0.05355 | | |
| | L-Tryptophan 0.00902 | | |
| | L-Tyrosine•2Na•2H₂O | | |
| | 0.05582 | | |
| | L-Valine 0.05285 | | |

In some embodiments of Hox media, the concentration of selenium ranges from about 2 μg/L to about 80 μg/L, e.g., 4 μg/L, 6 μg/L, 8 μg/L, 10 μg/L, 12 μg/L, 15 μg/L, 20 μg/L, 25 μg/L, 30 μg/L, 40 μg/L, 50 μg/L, 60 μg/L, 75 μg/L or another concentration of selenium from about 2 μg/L to about 80 μg/L. In one embodiment, the concentration of selenium is 14 μg/L.

In some embodiments, the concentration of insulin used in Hox media ranges from about 1 mg/L to about 50 mg/L, e.g., 2 mg/L, 3 mg/L, 5 mg/L, 7 mg/L, 8 mg/L, 10 mg/L, 15 mg/L, 20 mg/L, 25 mg/L, 35 mg/L, 40 mg/L, or another concentration of insulin from about 1 mg/L to about 50 mg/L. In one embodiment, the concentration of insulin is 19.4 mg/L.

As is known in the art, cell culture media should be buffered to a physiological pH of about 7.4. A number of agents suitable as pH buffers include, but are not limited to, bicarbonate, HEPES, TAPSO, or another Good's buffer suitable for buffering to a physiological pH of about 7.2 to about 7.6.

In some embodiments, Hox media also include, as a basal component, ascorbate. In some embodiments, the concentration of ascorbate used in the medium ranges from about 10 mg/L to about 200 mg/L, e.g., 15 mg/L, 25 mg/L, 30 mg/L, 40 mg/L, 50 mg/L, 60 mg/L, 75 mg/L, 80 mg/L, 100 mg/L, 125 mg/L, 150 mg/L, 175 mg/L, or another concentration of ascorbate from about 10 mg/L to about 200 mg/L. In one embodiment, the concentration of ascorbate is 64 mg/L.

In some embodiments, transferrin is included as a basal component. In some embodiments, transferrin can range in concentration from about 2 mg/L to about 50 mg/L, e.g., about 3 mg/L, 7 mg/L, 8 mg/L, 10 mg/L, 11 mg/L, 12 mg/L, 15 mg/L, 20 mg/L, 25 mg/L, 30 mg/L, 35 mg/L, 40 mg/L, or another concentration of transferrin from about 2 mg/L to about 50 mg/L. In one embodiment, the concentration of transferrin is 10.7 mg/L.

In other embodiments a Hox medium is substantially free of a TGFβ superfamily agonist (e.g., Nodal) and substantially free of an albumin. In some embodiments, the medium is also substantially free of putrescine or substantially free of progesterone. In other embodiments, the medium is substantially free of both putrescine and progesterone.

In some embodiments, the concentration of basal components in the medium will be as indicated in Table 2, except for one component, the concentration of which will fall within a range as described herein. In other embodiments, the concentration of more than one of the components can vary from that indicated in Table 2, but will fall within concentration ranges as described herein.

In one exemplary, non-limiting embodiment of a Hox medium, all the components listed in Table 1 are included (“Formulation 4” as listed in Table 1), and the concentrations of these components are those provided in Table 2.

In some embodiments, a Hox medium, referred to as a “Hox-Start” medium comprises a fully defined base medium as described above plus an activator of β-catenin pathway signaling and an FGF selected from among FGF2, FGF8a, FGF8b, FGF8f, FGF17, or FGF18 where the medium is substantially free of a TGFβ signaling activator (e.g., Nodal or TGFβ).

In some embodiments, particularly where induction of lumbar-Hox identity is desired (e.g., induction of Hox D10), the “Hox-Start” medium further comprises a growth differentiation factor (GDF), e.g., GDF11. In some embodiments, the medium including a GDF, also includes also includes an inhibitor of BMP signaling. Suitable inhibitors of BMP include, but are not limited to dorsomorphin, noggin, DMH1, and LDN193189. A suitable concentration of: dorsomorphin ranges from about 50 nM to about 1,000 nM (e.g., 200 nM); Noggin ranges from 25 ng/ml to about 400 ng/ml (e.g., 100 ng/ml); DMH1 ranges from about 20 nM to about 500 nM; and LDN193189 ranges from about 50 nM to about 1,000 nM.

A Hox-Start medium is useful to obtain Sox2⁺/Brachyury⁺/Pax6⁻/Otx2⁻ caudal lateral epiblasts and to initiate expression of rostral-caudal Hox gene expression starting from human pluripotent stem cells according to the methods described herein. Exemplary, non-limiting, basal media components and concentrations for Hox media, including Hox-Start media are shown in Tables 1 and 2. In some embodiments, the Hox-Start medium uses an alternative FGF, e.g., FGF2, FGF8a, FGF17b, FGF18, or a combination thereof.

In some embodiments, Hox-Start medium contains water, salts, amino acids, vitamins, a carbon source (e.g., glucose, fructose, sucrose, mannose, or galactose), a buffering agent, selenium, insulin, an FGF, an activator of the β-catenin pathway signaling, GDF11, and an inhibitor of BMP signaling, where the medium is substantially free of a TGFβ signaling activator. A Hox-Start medium is useful for initiating and driving rostral-caudal progression of Hox gene family expression in deriving caudal lateral epiblasts from human pluripotent stem cells by the methods disclosed herein.

In other embodiments, a Hox-Start medium may further include growth/differentiation factor 11 (GDF11), e.g., human, rat, or mouse GDF11. GDF11, a member of the BMP family of TGFβ superfamily proteins, is translated as an inactive preproprotein which is then cleaved and assembled into an active secreted homodimer. In some embodiments, where GDF 11 is included in a Hox-Start medium, the concentration of GDF 11 ranges from about 5 ng/ml to about 100 ng/ml, e.g., 10 ng/ml, 15 ng/ml, 20 ng/ml, 30 ng/ml, 50 ng/ml, 60 ng/ml, 75 ng/ml, or another concentration from about 5 ng/ml to about 100 ng/ml.

In other embodiments, a “Hox-Stop” medium comprises a fully defined neural differentiation base medium as described herein and in U.S. Patent Publication No. 2014/0134732, supplemented with a retinoid. “Hox-Stop” media are useful for arresting the rostral-caudal progression of Hox gene expression based on the timing of its application and the concentration of retinoid used as described herein. In some embodiments, where induction of lumbar/sacral identity is desired, a Hox-Stop medium also includes an inhibitor of BMP signaling.

As will be appreciated by those of ordinary skill in the art, β-catenin signaling can be activated by modulating the function of one or more proteins that participate in the β-catenin signaling pathway to increase β-catenin expression levels or activity, T-cell factor/lymphoid enhancer factor (TCF/LEF) expression levels, or β-catenin-TCF/LEF-mediated transcriptional activity.

In some embodiments, an activator of β-catenin pathway signaling is a small molecule that inhibits GSK3β phosophotransferase activity or GSK3β binding interactions. Suitable small molecule GSK3β inhibitors include, but are not limited to, CHIR 99021, CHIR 98014, BIO-acetoxime, BIO, LiCl, SB 216763, SB 415286, AR A014418, 1-Azakenpaullone, Bis-7-indolylmaleimide, and any combinations thereof in an amount or amounts effective to inhibit GSK3 phosophotransferase activity or GSK3 binding interactions. In some embodiments, any of CHIR 99021, CHIR 98014, and BIO-acetoxime are used to inhibit GSK3 in the differentiation methods described herein. In one embodiment, the small molecule GSK3β inhibitor used in a Hox Medium is CHIR99021 at a concentration ranging from about 1 μM to about 20 μM, e.g., about 2 μM, 3 μM, 4 μM, 5 μM 6 μM, 8 μM, 10 μM, 12 μM, 14 μM, 16 μM, or another concentration of CHIR99021 from about 1 μM to about 20 μM. In one embodiment, a Hox medium contains CHIR 99021 at a concentration of about 6 μM. In another embodiment, the small molecule GSK3 inhibitor to be used is CHIR 98014 at a concentration ranging from about 0.2 μM to about 2 μM, e.g., about 0.6 μM, 0.8 μM, 1 μM, 1.2 μM, 1.4 μM, 1.6 μM, or another concentration of CHIR98014 from about 0.2 μM to about 2 μM.

In other embodiments, a Hox medium contains an activator of β-catenin pathway signaling that acts by disrupting the interaction of β-catenin with Axin, a member of the β-catenin destruction complex. Disruption of Axin-β-catenin interaction allows β-catenin to escape degradation by the destruction complex thereby increasing the net level of β-catenin to drive β-catenin signaling. In some embodiments, a Hox medium contains 5-(Furan-2-yl)-N-(3-(1H-imidazol-1-yl)propyl)-1,2-oxazole-3-carboxamide (“SKL2001”), which is commercially available, e.g., as catalog no. 681667 from EMD4 Biosciences. An effective concentration of SKL2001 to activate β-Catenin signaling ranges from about 10 μM to about 100 μM, e.g., about 20 μM, 30 μM, 40 μM, 50 μM, 60 μM, 70 μM, 80 μM, 90 μM or another concentration of SKL2001 from about 10 μM to about 100 μM.

In further embodiments, a Hox-Start medium contains a Wnt polypeptide ligand, e.g., Wnt 3a. In some embodiments, the Hox-Start medium contains Wnt 3a at a concentration of about 10 ng/ml to about 400 ng/ml, e.g., 20 ng/ml, 30 ng/ml, 40 ng/ml, 50 ng/ml, 60 ng/ml, 80 ng/ml, 100 ng/ml, 120 ng/ml, 150 ng/ml, 170 ng/ml, 200 ng/ml, 250 ng/ml, 300 ng/ml, 350 ng/ml or another concentration of Wnt3a from about 20 ng/ml to about 400 ng/ml. In other embodiments, the Hox medium comprises any of Wnt 5a, Wnt 7a, Wnt 9b, and Wnt 10b.

Suitable retinoids and retinoid analogs for a Hox Stop medium include, but are not limited to All-Trans Retinoic Acid (ATRA), Retinol Acetate, and EC23 (4-[2-(5,6,7,8-Tetrahydro-5,5,8,8-te-tramethyl-2-naphthalenyl)ethynyl)-benzoic acid; CAS NO:. 104561-41-3)

In some embodiments, a Hox Stop medium contains ATRA. A suitable concentration of ATRA ranges from about 0.1 μM to about 20 μM, e.g., about 0.2 μM, 0.3 μM, 0.5 μM, 1.0 μM, 2.5 μM, 3.0 μM, 3.5 μM, 4.0 μM, 5 μM, 7 μM, 10 μM, 12 μM, 15 μM, 17 μM or another concentration of ATRA from about 0.1 μM, to about 20 μM. In some embodiments, the concentration of ATRA in the Hox Stop medium is about 3.0 μM. In some embodiments a suitable concentration of EC23 ranges from about 10 nM to about 200 nM, e.g., 20 nM, 30 nM, 50 nM, 80 nM, 100 nM, 120 nM, 150 nM, 180 nM, or another concentration from about 10 nM to about 200 nM.

In some embodiments, a Hox medium (start or stop formulations) is also substantially free of certain components. In some embodiments, a Hox medium is substantially free of a TGFβ pathway antagonist or BMP pathway antagonist. In other embodiments, a Hox medium, preferably a Hox-Start medium, contains a tumor growth factor β (TGFβ) signaling antagonist (e.g., SB431542, Sigma; at about 5-15 μM, e.g., 10 μM) and a bone morphogenetic protein (BMP) signaling antagonist (e.g., noggin at about 200 ng/ml or dorsomorphin at about 1 μM).

In some embodiments, a Hox-Start medium consists essentially of water, salts, amino acids, vitamins, a carbon source, a buffering agent, selenium, insulin, an FGF, an activator of β-catenin pathway signaling, GDF11, and an inhibitor of BMP signaling, where the FGF is selected from among FGF2, FGF8a, FGF8b, FGF8f, FGF17, and FGF18. In some embodiments, a Hox-Stop medium consists essentially of water, salts, amino acids, vitamins, a carbon source, a buffering agent, selenium, insulin, an activator of β catenin signaling, and a retinoid.

### Kits

In some embodiments described herein are kits comprising an FGF selected from among FGF2, FGF8a, FGF8b, FGF8f, FGF17, and FGF18 and an activator of β-catenin pathway signaling, which can be used to generate a Hox-Start medium as described herein. Optionally, the kit may further include a neural differentiation base medium for use in combination with the included supplements to generate a Hox-Start or Hox-Stop medium.

In some embodiments, the kit may also include a retinoid (e.g., retinol acetate or all-trans retinoic acid), which is useful for generating a Hox-Stop medium as described herein.

In some embodiments the kit also includes a GDF (e.g., GDF11).

In some embodiments, the kit includes, in addition to the GDF, an inhibitor of BMP signaling (e.g., dorsomorphin).

The concentration of each component in a kit may range from about five fold higher to about 200 fold higher than their final concentration in the neural differentiation medium, e.g., about 6, 10, 20, 30, 40, 50, 70, 80, 100, 120, 150, 180, or another fold higher than their final concentration in a Hox medium obtained by dilution of the concentrated component in a basal medium. In one embodiment, one or more of the kit components are at a 100 fold higher concentration than their final concentration after dilution in base medium, i.e., the concentrated components are at “100X” their final concentration in a Hox medium. In another embodiment, the concentrated components are supplied at a 50X concentration. In another embodiment, the concentrated components are supplied at a 200X concentration.

In some embodiments, FGF8b and the activator of β-catenin pathway signaling are provided together as a single concentrated supplement rather than as separate components.

In some embodiments the activator of β-catenin pathway signaling is a GSK3β inhibitor (e.g., CHIR 99021). In other embodiments the activator of β-catenin pathway signaling is Wnt3a.

### Cell-Based Compositions

An advantage of the media and methods described herein is the ability to specify the rostral-caudal axis identity of human caudal lateral epiblast cell populations, from an hPSC line, which give rise to similarly patterned posterior neuroectodermal, neuroepithelial cell, and human motor neuron populations. This is reflected in the ability to obtain essentially unlimited quantities of isolated populations of neuroectodermal cells, neuroepithelial cells, or motor neurons having a Hox gene mRNA expression profile characteristic of a distinct position along the rostral-caudal axis. In some embodiments, an isolated/enriched cell population has an mRNA expression profile in which Hoxa2 mRNA is found at a higher relative level than Hoxb4 mRNA. In other embodiments, an isolated cell population expresses Hoxa2 mRNA at a lower relative level than Hoxb4mRNA. In some embodiments, an isolated cell population expresses Hoxb4 mRNA at a higher relative level than Hoxc6 mRNA. In other embodiments, an isolated cell population expresses Hoxb4 mRNA at a lower relative level than Hoxc6 mRNA. In some embodiments, an isolated cell population expresses Hoxc6 mRNA at a higher relative level than Hoxc9 mRNA. In other embodiments, an isolated cell population expresses Hoxc6 mRNA at a lower relative level than Hoxc9 mRNA. In some embodiments, an isolated cell population expresses Hoxc9 mRNA at a higher relative level than Hoxd10 mRNA. In other embodiments, an isolated cell population expresses Hox d10 mRNA at a higher relative level than Hox c9 mRNA.

In other embodiments, an isolated cell population, as a whole, expresses Hoxb4 mRNA at a higher relative level than Hoxd10 mRNA. In some embodiments, the relative expression level of a first Hox gene mRNA (e.g., Hoxb4) in an isolated population of human caudal lateral epiblast, neuroectodermal, neuroepithelial cells (NECs), or motor neurons is about two fold to about 50 fold higher than a second hox gene (e.g., HoxdlO), e.g., about 3 fold, 4 fold, 5 fold, 6 fold, 10 fold, 12 fold, 15 fold, 20 fold, 30 fold, 40 fold, or another fold higher from about two fold to about 50 fold higher. Such isolated populations are obtained according to the differentiation methods described herein. One of ordinary skill in the art will appreciate that when comparing mRNA expression levels of various Hox genes to one another, the absolute level of each Hox gene mRNA is less important than the relative level. As referred to herein, a “relative mRNA expression level” for a given Hox gene refers to a mRNA expression value that is normalized to a sample having the maximum expression level for that Hox gene within a given sample set, where the maximum expression level is set to a value of 1. In some cases, the relative expression levels are normalized, as just mentioned, relative to a standard mRNA reference sample. The mRNA standard sample contains a maximum (reference) amount of a Hox gene mRNA (e.g., Hoxd10 mRNA). Use of a standard reference sample then allows Hox gene mRNA expression samples to be normalized. For example, if measuring the level of Hoxb4 and Hoxd10 in a sample shows that the normalized expression values of Hoxb4 and Hox d10 are 0.25 and 0.75, respectively, than the relative expression level of Hoxb4 is stated to be three fold lower than that of Hoxd10.

In some embodiments, at least 50% of the cells in an isolated population of human caudal lateral epiblast, neuroectodermal, or neuroepithelial cells comprises one of the above-described Hox gene mRNA profiles, e.g., 55%, 60%, 65%, 70%, 75%, 80%, 85%, 90%, 95%, 99%, or another percent of cells in the isolated cell population exhibit the desired Hox gene mRNA profile. Methods for enriching cell populations based on mRNA expression are known in the art. For example, molecular beacon probes can be used to purify cells expressing a specific mRNA or set of mRNAs. See, e.g., Ban et al (2013), Circulation (published online Aug. 30th, 2013; PMID: 23995537).

mRNA expression levels in a population can be detected using any of a number of routine methods in the art including, but not limited to, qRT-PCR, RNA-blot, RNA sequencing, and RNAse protection.

In some embodiments described herein is a cell culture that includes any of the just described isolated human caudal lateral epiblast populations, and a Hox-Stop medium. In some embodiments, the Hox medium contains water, salts, amino acids, vitamins, a carbon source, a buffering agent, selenium, and insulin.

In some embodiments the isolated cell populations described herein comprise cells (e.g., caudal lateral epiblasts, neuroectodermal cells, neuroepithelial cells, or motor neurons) that are genetically modified cell populations. For example, the cell populations can be obtained by differentiation of a genetically modified hPSC line (e.g., a transgenic line, a “knock-in” line, or a “knock-out” line). Methods for establishing genetically modified hPSC lines are well known in the art. See, e.g., Sun et al (2012), Biotechnol J., 7(9):1074-1087; and Chatterjee et al (2011), 5;(56); pg. 3110. Alternatively, the isolated cell populations can be genetically modified directly by transient transfection (e.g., transfection of plasmid expression vectors, oligonucleotides, RNAi, or modified mRNAs) or viral transduction. In some embodiments such cells are genetically modified with an expression cassette or exogenous RNA encoding a fluorescent reporter protein, a growth factor, an extracellular protein, or an antibody.

### IV. Methods

In various embodiments, the differentiation and specification of hPSCs into caudal lateral epiblasts is effected by culturing the PSC using various media in combination with the additives and timing regimen described herein.

In some embodiments, a method for generating caudal lateral epiblasts from hPSCs includes the steps of: (i) culturing human pluripotent stem cells during a first culture period of about one to two days with a neural differentiation base medium to obtain a first cell population; (ii) culturing the first cell population for a second culture period of about one day to about four days in neural differentiation base medium supplemented with an FGF to obtain a second cell population that is Sox2⁺, Otx2⁺, Brachyury⁻, and Pax6⁻; and (iii) culturing the second cell population for a third culture period of about one day to about seven days in neural differentiation base medium comprising an FGF and an activator of β-catenin pathway signaling to obtain caudal lateral epiblasts that are Sox2⁺/Brachyury⁻/Pax6⁻/Otx2⁻, wherein the FGF is FGF2, FGF8a, FGF8b, FGF8f, FGF17, or FGF18.

In some embodiments, the third culture period is at least six days, wherein after at least four days of this third culture period, the culture medium to be used also includes GDF11 and an inhibitor of BMP signaling.

In some embodiments the third culture period is for about 24 hours to about 42 hours, wherein caudal lateral epiblasts obtained express Hoxb4 mRNA at a higher level than Hoxc6 mRNA. In other embodiments the third culture period is for about 60 hours to about 84 hours, wherein Hoxc6 mRNA is expressed at a higher level than Hoxb4 mRNA or Hoxc9 mRNA. In other embodiments the third culture period is for about 96 hours to about 144 hours, wherein Hoxc9 mRNA is expressed at a higher than Hoxc6 mRNA. In some embodiments the third culture period is for about 156 hours to about 170 hours, wherein Hoxd10 mRNA is expressed at a higher level than Hoxc9 mRNA.

In various embodiments, hPSCs, e.g., hESCs or hiPSCs, are cultured in the absence of a feeder layer (e.g., a fibroblast layer) on a substrate suitable for proliferation of hPSCs, e.g., MATRIGEL®, vitronectin, a vitronectin fragment, or a vitronectin peptide, or SYNTHEMAX®, prior to plating for differentiation into posterior neuroectoderm. In some cases, the hPSCs are passaged at least 1 time to at least about 5 times in the absence of a feeder layer. Suitable serum-free culture media for passaging and maintenance of hPSCs include, but are not limited to, MTESR® and E8™ medium). In some embodiments, the hPSCs are maintained and passaged under xeno-free conditions, where the cell culture medium is a defined medium such as E8™ or MTESR®, but the cells are maintained on a completely defined, xeno-free substrate such as vitronectin, or SYNTHEMAX® (or another type-of self-coating substrate).

In one embodiment, the hPSCs are maintained and passaged in E8™ medium on vitronectin, a vitronectin fragment, or a vitronectin peptide or a self-coating substrate such as SYNTHEMAX®.

Typically, to increase single cell plating efficiency and cell viability hPSCs are initially plated on one of the above-mentioned feeder-free substrates in one of the above-mentioned media in the presence of a Rho-Kinase (ROCK) inhibitor, e.g., Y-27632 (R&D Systems) at a concentration of about 10 μM and cultured overnight prior to initiating neural differentiation.

Any serum-free culture medium suitable for passaging and maintenance of hPSCs can be used in combination with any substrate suitable for proliferation of hPSCs in the absence of feeder cells.

The density of hPSCs is an important factor affecting the efficiency of the caudal lateral epiblast differentiation methods described herein. In preparation for caudal lateral epiblast differentiation as described herein, hPSCs are typically plated at a density of at least about 1×10⁵ cells/cm² to about 2×10⁵ cells/cm², whereby the cells will be at least about 95% confluent upon changing the medium from one suited for hPSC proliferation to one that initiates differentiation of the hPSCs as described herein. Typically, the hPSCs will be maintained in a pluripotent stem cell maintenance medium (e.g., “E8” medium) as described above for a period of about 1 day or until the hPSCs are at least about 95% confluent.

Starting on “Day 0” hPSCs are incubated and cultured in a serum-free medium that supports neural differentiation “neural differentiation base medium.” Suitable neural differentiation media are also described in U.S. Patent Publication No. 2014/0134732. Typically the hPSCs are cultured in the neural differentiation base medium for a period of about 24 hours to obtain a first cell population characterized by Sox2 and Otx2 protein expression and the absence of Brachyury, Sox17, and Pax6 protein expression.

In some embodiments, the neural differentiation base medium to be used in the neural differentiation method consists essentially of a base medium (e.g., DMEM/F12 or a similar base medium as described herein) containing water, salts, amino acids, vitamins, a carbon source, and a buffering agent; plus the supplemental components selenium and insulin (referred to as “E4 medium”). In some embodiments, the neural differentiation base medium to be used includes, in addition to the components listed for E4 medium, ascorbate to generate a medium referred to herein as an “E5 medium”. In a preferred embodiment the differentiation medium to be used is a carbonate-buffered E5 medium plus transferrin, which is also referred to herein as an “E6 medium”.

In some embodiments, the medium to be used does not include a transforming growth factor β (TGFβ) signaling antagonist or a bone morphogenetic protein (BMP) signaling antagonist. In other embodiments, the medium to be used is an E4 medium in combination with a transforming growth factor β (TGFβ) signaling antagonist (e.g., SB431542, Sigma; at about 10 μM) and a bone morphogenetic protein (BMP) signaling antagonist (e.g., noggin at about 200 ng/ml or dorsomorphin at about about 1 μM).

In some embodiments, directed differentiation of human pluripotent stem cells into neural stem cells, is carried out by culturing pluripotent stem cells on a substrate that supports proliferation of pluripotent stem cells (e.g., vitronectin or MATRIGEL®), and in a serum-free medium comprising water, salts, amino acids, vitamins, a carbon source, a buffering agent, selenium and insulin, wherein human pluripotent stem cells are cultured in the substantial absence of any of embryoid bodies, a TGFβ superfamily agonist, a TGFβ signaling antagonist, or a BMP signaling antagonist.

After a culture period in neural differentiation base medium, the resulting first cell population is then cultured in a neural differentiation base medium containing FGF8b at a concentration of about 50 ng/ml to about 400 ng/ml, e.g., 60 ng/ml, 70 ng/ml, 100 ng/ml, 120 ng/ml, 150 ng/ml, 170 ng/ml, 200 ng/ml, 250 ng/ml, 300, ng ml, 375 ng ml, or another concentration of FGF8b ranging from about 50 ng/ml to about 400 ng/ml. Suitable FGF8bs to be used include those from human (GenBank Accession No. NP_{—}006110) or mouse (GenBank Accession No. NP_{—}001159834). In some embodiments, a naturally occurring or artificial homolog of FGF8b having at least 75% identity to the above mentioned human or mouse amino acid sequences is used instead, e.g., a homolog having 80%, 85%, 90%, 95%, 97%, 99%, or another percent amino acid sequence identity to FGF8b ranging from at least 75% to 100% identical. For example, in some cases human FGF17 (GenBank Accession No. O60258.1) may be used instead of FGF8b within a similar concentration range. In other embodiments, any of FGF2, FGF8A, FGF17B, FGF18, or a combination thereof is used. In some embodiments, the first cell population is cultured for a period of about 24-96 hours to obtain a second population characterized by expression of Sox2 and Otx2, and the absence of Brachyury, Sox17, and Pax6.

Subsequently, the second population of cells obtained after culture in the presence of FGF8b is cultured during a third culture period in neural differentiation base medium containing FGF8b and an activator of β-catenin pathway signaling (Hox-Start medium) for a period of about one day to about seven days, e.g., about two days, three days, four days, five days, six days or another period from about one day to about seven days. In some embodiments, this third culture period lasts about one day. This culture period results in the generation of a caudal lateral epiblast cell population characterized by expression of Sox2 and Brachyury (AKA “T”), but no PAX6 expression, and by a pattern of Hox expression that depends on the length of exposure to FGF8b and the activator of β-catenin pathway signaling. Shorter culture periods yield a more rostral Hox gene expression pattern where, for example, the expression of Hoxb4 is expressed at a higher level than Hoxd10, and, conversely, where longer culture periods yield a caudal lateral epiblast cell population that expresses a more caudal Hox expression pattern, e.g., with higher level of Hoxd10 than Hoxb4 expression. This caudal lateral epiblast cell population is also characterized by expression of Sox2 and Brachyury and the absence of PAX6 expression.

Suitable activators of β-catenin pathway signaling for use in the methods described herein include any of those described herein for the generation of a Hox-Start medium. In some embodiments, the activator of β-catenin pathway signaling used is a GSK3β kinase inhibitor. In one embodiment, the GSK3β kinase inhibitor used is CHIR99021 at a concentration ranging about 1 μM to about 20 μM. In one embodiment, a Hox medium contains CHIR 99021 at a concentration of about 3 μM.

While not wishing to be bound by theory, it is believed that culture of caudal lateral epiblast in the presence of a retinoid converts the lateral epiblast to Sox2⁺/Pax6⁺ neuroectoderm that spontaneously polarizes into neuroepithelium, as indicated by an asymmetric distribution of intramembranous N-cadherin, and “fixes” the Hox expression in the resulting posterior neuroectoderm based on the timing of retinoid addition to the patterned caudal lateral epiblasts. Thus, the later a retinoid is added relative to the initial generation of caudal lateral epiblasts from hPSCs, the more caudal the Hox expression pattern will be in the resulting posterior neuroectoderm and neuroepithelium.

In order to generate PAX6⁺ neuroepithelium characterized by a specified and relatively uniform Hox gene expression pattern, a Sox2⁺/Brachyury⁻/Pax6⁻ caudal lateral epiblast cell population, obtained as described above, is cultured in a neural differentiation base medium in the presence of a retinoid (Hox-Stop medium) for a period of about one to five days, e.g., two days, three days, four days days, or another period from about one day to about five days. In some embodiments, the fourth culture period is about four days. Suitable retinoids for use in this method include any of those described for generating a Hox-Stop medium, e.g., ATRA or EC23. In some embodiments, the retinoid to be used is All-Trans Retinoic Acid (ATRA). In one embodiment, ATRA is used at a concentration of about 1 μM.

In some embodiments, posterior neuroectoderm or neuroepithelium from hPSCs is generated by a method that includes: culturing a population of Sox2^{|}/Brachyury^{|}/PAX6⁻/Otx2⁻ human caudal lateral epiblasts that expresses at least one Hox gene in a neural differentiation base medium comprising a retinoid to obtain posterior neuroectoderm or neuroepithelium comprising a population of cells that are Sox2⁺/PAX6⁺/Brachyury⁻ and express at least one Hox gene, where the neural differentiation base medium comprises water, salts, amino acids, vitamins, a carbon source, a buffering agent, selenium, and insulin.

Also described herein is a method for generating a population of human motor neurons having a specified Hox gene expression profile, comprising culturing human posterior neuroectoderm or neuroepithelium having a specified Hox gene expression profile, as described above, in a neural differentiation base medium supplemented with a retinoid and an activator of the Hedgehog signaling pathway to obtain the population of human motor neurons. The Hox identity (e.g., lumbar) is determined at the lateral epiblast stage, and fixed upon exposure to retinoic acid to obtain neuroectoderm as described herein. Accordingly, populations of human motor neurons having pre-determined Hox identity can be generated by further differentiation of neuroectoderm having the desired Hox identity.

Suitable activators of the hedgehog pathway include, but are not limited to purmorphamine (at a concentration from about 25 nM to about 300 nM), smoothened agonist (SAG), or CUR61414.

In some embodiments the human caudal lateral epiblasts to be used in the above method express Hoxd10. In some embodiments the human posterior neuroectoderm cells express Hoxc9. In other embodiments the human posterior neuroectoderm cells express Hoxb4. In further embodiments, the human posterior neuroectoderm cells express Hoxa2. As described herein, expression levels of a set of Hox gene mRNAs are compared on the basis of normalized Hox gene expression mRNA levels.

Suitable quantitative methods for evaluating any of the above-markers are well known in the art and include, e.g., qRT-PCR, RNA-sequencing, RNA-blot, RNAse protection, and the like for evaluating gene expression at the RNA level. Quantitative methods for evaluating expression of markers at the protein level in cell populations are also known in the art. For example, flow cytometry, is typically used to determine the fraction of cells in a given cell population expressing (or are not expressing) one or two protein markers of interest (e.g., PAX6, Brachyury, and Sox2).

In sum, we disclose herein that Wnt/β-catenin and FGF signaling synergistically control HOX activation while maintaining a neuromesodermal progenitor state, while using a retinoic acid receptor agonist such as RA arrests HOX activation to yield a fixed rostrocaudal position and transitions the cultures to definitive neuroectoderm. In addition, while Wnt/β-catenin and FGF activated HOXparalog expression from hindbrain through thoracic identity, GDF11 supplementation was required to facilitate lumbosacral patterning. as expected from in vivo. The disclosed methodology facilitates the generation of neural cells from hPSCs corresponding to any rostrocaudal position spanning the hindbrain to lumbosacral spinal cord in a predictable, deterministic manner. These findings are distinct from other hPSC neural differentiation systems, which have generated neural cells possessing heterogeneous rostrocaudal identity.

The invention will be more fully understood upon consideration of the following non-limiting Examples.

### EXAMPLES

### Example 1

### Specification of HOX-Expressing Posterior Caudal Lateral Epiblast from hPSCs

During formation of the hindbrain and spinal portions of the neural tube, it is currently believed that signaling by FGFs and Wnts maintain an undifferentiated, Sox2⁻/Pax6⁻ caudal lateral epiblast stem-like phenotype in the posterior neural tube, whereas retinoic acid (RA) secreted from newly formed somites in the paraxial mesoderm counteracts such signaling to force differentiation to a Sox2⁺/Pax6⁺ neuroectoderm or neuroepithelial state. Moreover, since forebrain explants exposed to FGFs and Wnts can be re-specified to Hox expressing neural tissue, we hypothesized that these morphogens can be used to induce Hox gene expression as hPSCs differentiate into caudal lateral epiblasts. Further, since RA signaling in the neural tube occurs upon regression of the node and concurrent somitogenesis, it can be hypothesized that there is temporal variation in the duration of FGF and Wnt exposure experienced by caudal lateral epiblasts prior to RA-induced neuroectodermal differentiation. Thus, we also hypothesized that the duration of FGF and Wnt exposure affects Hox expression, and RA exposure could be used to counteract the effects of FGFs and Wnts.

### Methods.

hPSC maintenance. hPSCs were maintained E8 medium¹ consisting of DMEM/F12 (Invitrogen), 64 mg/L ascorbic acid (Sigma), 543 mg/L sodium bicarbonate (Sigma), 14 μg/L sodium selenite (Sigma), 19.4 mg/L insulin (Sigma), 10.7 mg/L transferrin (Sigma), 100 μg/L FGF2 (Waisman Clinical Biomanufacturing Facility, University of Wisconsin-Madison), and 2 μg/L TGFβ1 (Peprotech). pH of E8 medium was adjusted to 7.4 and osmolarity was adjusted to 340 mOsm with NaCl. hPSCs were maintained on MATRIGEL® (BD Biosciences) or recombinant vitronectin peptide (VTN-NC; provided by Dr. James Thomson and described in U.S. Patent Publication No. 2012/0301962). VTN-NC-coated plates were pre-coated with 100 μg/ml poly-L-ornithine (Sigma) for 1 h and washed twice with sterile ddH₂O before coating with VTN-NC (8 μg/well) overnight at 37° C. Cell lines used in this study were H9 hESCs (passage 25-50). Cells were routinely passaged with Versene (Invitrogen).

hPSC differentiation. hPSCs were washed once with phosphate-buffered saline (PBS; Invitrogen), dissociated with ACCUTASE® (Invitrogen) for 3 min, and collected by centrifugation. hPSCs were then seeded onto VTN-NC-coated plates in E8 medium containing 10 μM ROCK inhibitor (Y27632; R&D Systems). Seeding density was 2×10⁵ cells/cm². The following day, the cells were switched to E6 medium (same composition as E8 medium minus FGF2 and TGFβ1). Morphogen treatments were then carried out according to the figures in the text. Cells were supplemented with FGF8b (100-200 ng/ml; Peprotech), CHIR99021 (3-6 μM; Tocris), and/or RA (3 μM; Sigma).

Reverse transcriptase polymerase chain reaction (RT-PCR). Total RNA was extracted from cells using TRIZOL® reagent (Invitrogen) according to the manufacturer's instructions. RNA was then subjected to reverse-transcription using a Thermoscript™ RT-PCR kit (Invitrogen) in a 20 μL mixture according to the manufacturer's instructions. The resulting cDNA was then amplified in a 25 μL mixture containing 10× PCR buffer, 0.2 mM dNTP, 1.5 mM MgCl₂, 0.5 μM of each primer, and 1 U Taq DNA polymerase (Invitrogen). Amplified products were resolved on 2% agarose gels containing SYBR Safe (Invitrogen) and visualized with a VersaDoc (BioRad). Primer sequences can be found in Table 3. All amplifications were carried out for 35 cycles. HOXC5 and HOXC6 were amplified at 58° C., all others with an annealing temperature of 55° C.

TABLE 3
Primer sequences used for RT-PCR

| Gene | Primer sequence |
|---|---|
| GAPDH | F: CACCGTCAAGGCTGAGAACG (SEQ ID NO: 1) |
| | R: GCCCCACTTGATTTTGGAGG (SEQ ID NO: 2) |
| SOX2 | F: TGGACAGTTACGCGCACAT (SEQ ID NO: 3) |
| | R: CGAGTAGGACATGCTGTAGGT (SEQ ID NO: 4) |
| PAX6 | F: GGCAACCTACGCAAGATGGC (SEQ ID NO: 5) |
| | R: TGAGGGCTGTGTCTGTTCGG (SEQ ID NO: 6) |
| T | F: CTTCCCTGAGACCCAGTTCA (SEQ ID NO: 7) |
| | R: CAGGGTTGGGTACCTGTCAC (SEQ ID NO: 8) |
| OTX2 | F: CCAGACATCTTCATGCGAGAG (SEQ ID NO: 9) |
| | R: GGCAGGTCTCACTTTGTTTTG (SEQ ID NO: 10) |
| HOXA1 | F: AAATCAGGAAGCAGACCCAC (SEQ ID NO: 11) |
| | R: GTAGCCGTACTCTCCAACTTTC (SEQ ID NO: 12) |
| HOXB4 | F: TACCCCTGGATGCGCAAAGTTC (SEQ ID NO: 13) |
| | R: TGGTGTTGGGCAACTTGTGG (SEQ ID NO: 14) |
| HOXC5 | F: ACAGATTTACCCGTGGATGAC (SEQ ID NO: 15) |
| | R: AGTGAGGTAGCGGTTAAAGTG SEQ ID NO: 16) |
| HOXC6 | F: GAATGAGGGAAGACGAGAAAGAG |
| | (SEQ ID NO: 17) |
| | R: CATAGGCGGTGGAATTGAGG (SEQ ID NO: 18) |
| HOXC8 | F: TTTATGGGGCTCAGCAAGAGG (SEQ ID NO: 19) |
| | R: TCCACTTCATCCTTCGGTTCTG (SEQ ID NO: 20) |
| HOXC9 | F: AGCACAAAGAGGAGAAGGC (SEQ ID NO: 21) |
| | R: CGTCTGGTACTTGGTGTAGG (SEQ ID NO: 22) |
| HOXC10 | F: AAAGGAGAGGGCCAAAGC (SEQ ID NO: 23) |
| | R: GCGTCTGGTGTTTAGTATAGGG (SEQ ID NO: 24) |

Flow cytometry. Cells were washed once with PBS, dissociated with ACCUTASE® for 5 min, and collected by centrifugation. After fixation in 4% paraformaldehyde for 10 min at room temperature, blocking/permeabilization was conducted with 10% donkey serum (Sigma) in PBS (10% PBSD) containing 0.1% Triton X-100 (Fisher Scientific) for at least 30 min at room temperature. Cells were then resuspended in 100 μL of 10% PBSD containing primary antibody and incubated overnight at 4° C. Antibodies are listed in Table 4. The following day, cells were washed twice with PBS containing 1% bovine serum albumin (BSA) and incubated with secondary antibodies diluted in 10% PBSD for 45 min at room temperature. Following another two washes, cells were analyzed on a FACSCalibur™ (BD Biosciences). Data analysis was conducted with Cyflogic software.

Table 4
Antibodies used for flow cytometry (FC) and immunocytochemistry (ICC).

| | | | Clone | |
|---|---|---|---|---|
| Target antigen | Antibody species | Vendor | (if applicable) | Dilution |
| Sox2 | mouse | Millipore | 10H9.1 | 1:1000 (FC) |
| | | | | 1:200 (ICC) |
| Pax6 | mouse | DSHB | N/A | 1:200 (FC) |
| Pax6 | rabbit | Covance | N/A | 1:500 (ICC) |
| N-cadherin | mouse | BD Biosciences | 32 | 1:500 (ICC) |
| Otx2 | goat | R&D Systems | N/A | 1:200 (FC) |
| brachyury | goat | R&D Systems | N/A | 1:200 (FC) |
| | | | | 1:500 (ICC) |
| HoxB4 | rat | DSHB | N/A | 1:50 (ICC) |
| Donkey anti-rabbit | N/A | Invitrogen | N/A | 1:200 (FC) |
| Alexa Fluor 488 | | | | 1:500 (ICC) |
| Donkey anti-mouse | N/A | Invitrogen | N/A | 1:200 (FC) |
| Alexa Fluor 488 | | | | 1:500 (ICC) |
| Donkey anti-goat | N/A | Invitrogen | N/A | 1:200 (FC) |
| Alexa Fluor 488 | | | | 1:500 (ICC) |
| Donkey anti-mouse | N/A | Invitrogen | N/A | 1:200 (FC) |
| Alexa Fluor 647 | | | | 1:500 (ICC) |
| Donkey anti-rabbit Cy3 | N/A | Jackson | N/A | 1:200 (FC) |
| | | Immunoresearch | | 1:500 (ICC) |
| Donkey anti-mouse Cy3 | N/A | Jackson | N/A | 1:200 (FC) |
| | | Immunoresearch | | 1:500 (ICC) |
| Donkey anti-rat Cy3 | N/A | Jackson | N/A | 1:200 (FC) |
| | | Immunoresearch | | 1:500 (ICC) |

Immunocytochemistry. Cells were washed twice with PBS and fixed in 4% paraformaldehyde for 10 min at room temperature, followed by three additional washes with PBS. Cells were then blocked and permeabilized in tris-buffered saline (TBS) containing 5% donkey serum and 0.3% Triton X-100 (TBS-DT) for at least 1 h at room temperature. Primary antibodies (Table 4) were then diluted in TBS-DT and incubated on the cells overnight at 4° C. The following day, cells were washed five times with TBS containing 0.3% Triton X-100 and then incubated with TBS-DT containing secondary antibodies for 1-2 h at room temperature. Nuclei were subsequently counterstained with 300 nM 4′,6-Diamidino-2-pheny-lindoldihydrochloride (DAPI; Invitrogen) for 10 min. After five washes with TBS, cells were imaged with a Nikon Ti-E microscope. Nikon NIS-Elements software was used for image analysis.

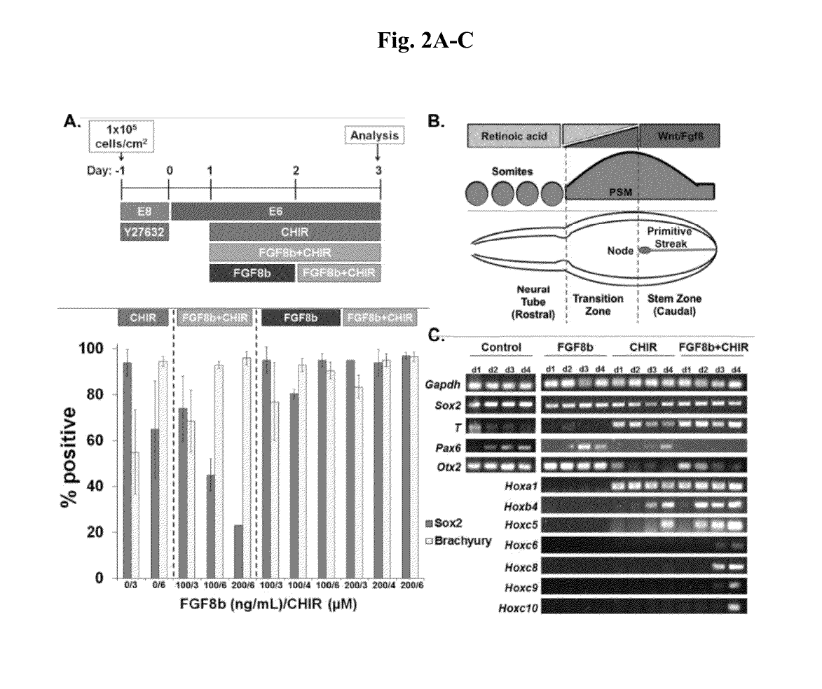

We performed time course HOX expression analysis of hPSCs being differentiated into caudal lateral epiblast via exposure to Fgf8b (100 ng/ml) and CHIR 99021 (3 μM) (FIG. 2C). We observed that Wnt/β-catenin signaling (i.e. CHIR 99021) alone could induce Hox gene expression, whereas Fgf8b exposure could not; however, the application of both Fgf8b and CHIR 99021 could pattern cells even further down the neuraxis to yield expression of Hoxc10 by the fourth day of combined FGF8b/CHIR 99021 exposure. Thus, it appeared that FGF8b exposure accelerated activation of the Hox clusters by Wnt signaling. Interestingly, the HOX genes appear to be activated in a periodic sequential fashion, not all at once. This is reminiscent of collinear Hox expression during CNS development, where 3′-Hox genes (e.g. Hoxal and Hoxb4) are expressed first in the hindbrain while 5′-HOX genes (Hoxc9 and Hoxc10) are expressed later in the lumbosacral spinal cord (FIG. 1). This sequential HOX expression also correlates with recent observations made using chromatin conformation capture techniques, which show the HOX loci sequentially transitioning from a transcriptionally-inactive chromosomal compartment to an active one upon expression during development. Therefore, we believe our patterning protocol recapitulates developmental processes that regulate HOX gene activation in the neural tube. To our knowledge, this was the first explicit demonstration of temporal activation of HOX genes in neurally differentiating hPSCs.

### Example 2

### Sequential Treatment of hPSCs with FGF8b and an Activator of β-Catenin Pathway Signaling Yields a Sox2⁺/Brachyury⁺/Pax6⁻ Caudal Lateral Epiblast Intermediate

In posterior neural tube development, caudal lateral epiblasts initially express both the neuroectoderm marker Sox2 and the mesoderm marker Brachyury (T). Since these cells form HOX-expressing portions of the neural tube, we hypothesized that they would be sensitive to morphogenetic cues that pattern HOX genes, and thus attempted to derive such cells from hPSCs. Signaling via both Fibroblast growth factor-8 (Fgf8) and Wnts has been shown to play a role in formation of the posterior neural tube. We therefore exposed H9 (WA09) hPSCs cultured in E6 medium supplemented with recombinant Fgf8b and CHIR 99021 early in our E6 neural differentiation method. From flow cytometry analysis, we observed that sequential addition of Fgf8b and then Fgf8b/CHIR 99021 yielded a nearly homogenous culture of Sox2⁺/Brachyury⁺ caudal lateral epiblast (FIG. 2A). To our knowledge, this is the first explicit protocol for deriving Sox2⁺/Brachyury⁺/PAX6⁻/Otx2⁻ caudal lateral epiblasts from hPSCs.

### Example 3

### Progression of HOX Gene Expression In Vitro can be Controlled During Differentiation of hPSCs to Caudal Lateral Epiblasts

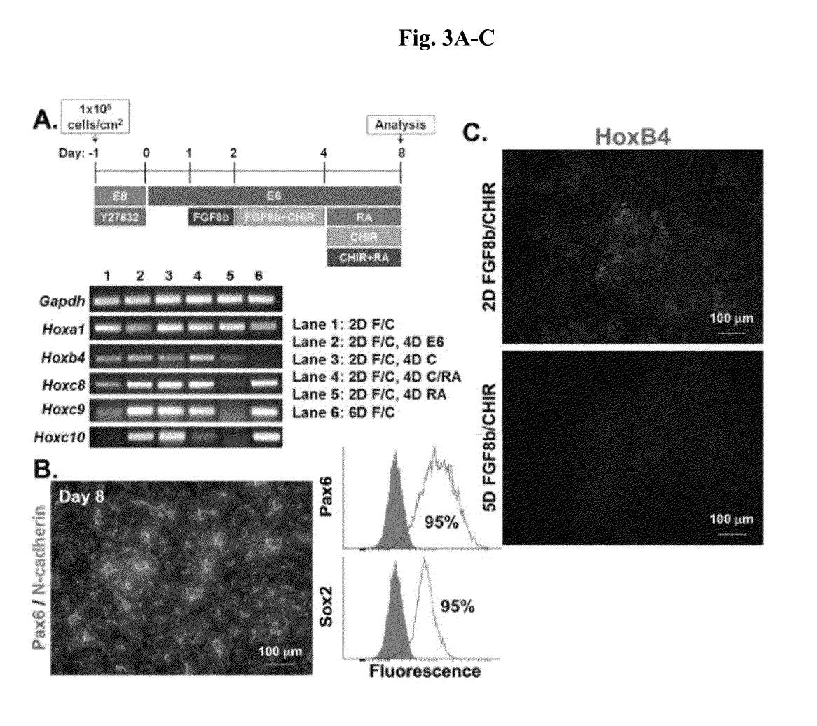

In order to determine whether the progression of HOX gene activation is morphogen concentration-dependent, and whether it can be halted at the onset of expression of specific HOX genes, we performed the same experiment as described in Example 1, but at higher morphogen concentrations followed by multiple culture variations to inhibit the effects of FGF8b and CHIR 99021 (FIG. 3A). After 48 hours of Fgf8b (200 ng/m1) and CHIR 99021 (4 μm) treatment, HOX gene activation in the caudal lateral epiblasts had reached the Hoxc9 locus as opposed to reaching just Hoxc5 under the lower morphogen conditions (FIGS. 2C and 3A), thereby indicating that the rate of Hox gene activation may be morphogen concentration-dependent. Additionally, we demonstrated that removal of Fgf8b and CHIR 99021 and addition of RA is sufficient to halt Hox gene activation, as observed by the lack of Hoxc9 and Hoxc10 expression in Lane 5 but in none of the other lanes (FIG. 3A). Further, we observed that caudal lateral epiblast cultures patterned by FGF8b and CHIR 99021 and halted by RA exposure could differentiate to definitive caudal neuroepithelium characterized by Pax6 expression and polarization of N-cadherin (FIG. 3B).

As a final proof-of-principle, we demonstrated by immunocytochemistry that caudal lateral epiblasts patterned for two versus five days of Fgf8/CHIR 99021 before RA-treatment expressed HoxB4 protein, a marker associated with more rostral positions, whereas the more caudally patterned cells (i.e. the five day Fgf8/CHIR 99021 group) were not positive for HoxB4 protein expression (FIG. 3C). This indicates that the trend observed with HOX gene activation by RT-PCR is also present at the protein expression level, where Hox protein expression also varies in a temporally collinear fashion. To our knowledge, this level of control over HOX expression in neurally differentiating hPSCs has never been demonstrated before.

### Example 4

### Deterministic HOX Patterning in hPSC-Derived Posterior Neuroectoderm

In all vertebrates, colinear activation of HOX genes confers positional information across many developing tissue structures from all embryonic germ layers¹. Despite observations of HOX expression in human pluripotent stem cell (hPSC)-derived progeny²⁻⁵, the ability to predictably control HOX expression patterns and generate pure populations of regionally specified cells defined by combinatorial HOX expression profiles has not been demonstrated, which limits basic biological studies and potential regenerative medicine applications. In this Example, we describe a fully defined hPSC differentiation system that deterministically controls collinear HOX expression in highly pure Sox2⁺/Brachyury⁺ neuromesodermal progenitors by manipulating Wnt/β-catenin, fibroblast growth factor (FGF), and growth differentiation factor (GDF) signaling. Retinoic acid (RA) treatment transitions these neuromesodermal progenitors (also referred to herein as caudal lateral epiblasts) to definitive neural identity while halting HOX progression to yield distinct hindbrain, cervical, thoracic, and lumbar/sacral neural progenitors defined by combinatorial Hox protein expression. Taken together, this work represents a detailed blueprint for generating human neural cells at any prospective position along the posterior neuroaxis. Moreover, these mechanisms are relevant to controlling HOX expression in other hPSC-derived lineages.

Materials and Methods

Propagation and differentiation of neuromesodermal progenitors. H9 hESCs (passage 25-45), H9 ishcat2 hESCs (passage 33-43), and IMR90-4 iPSCs (passage 31-40) were maintained in E8 medium on MATRIGEL® (BD Biosciences) as previously described. To initiate differentiation, hPSCs were passaged with ACCUTASE® (Life Technologies) onto vitronectin (VTN-NC)-coated plates at a density of 1×10⁵ cells/cm² as previously described. The following day, cells were changed to E6 medium. 24 h later, cells were changed to E6 medium containing 200 ng/ml FGF8b (Peprotech). 24 h later, cells were washed once with 2 ml PBS, treated with ACCUTASE® for 2 min, and removed from the surface by gentle pipetting. After collection by centrifugation, cells were gently resuspended in E6 medium containing 200 ng/ml FGF8b and CHIR99021 (CHIR, concentration varied depending on cell line; Tocris) and re-seeded on VTN-NC-coated plates at a density of 1.5×10⁵ cells/cm². Medium was not changed until 48 h after passaging. This passaging process was repeated on day 3 of CHIR 99021 treatment (re-seed density of 1.2×10⁵ cells/cm²) if extended neuromesodermal propagation was required, and 50 ng/ml GDF11 (Peprotech) was added on day 4 of CHIR 99021 treatment to initiate lumbar patterning. Cells were changed to E6 medium containing 1 μM retinoic acid (RA; Sigma) to facilitate neural transition.

For assessment of columnar identity by FoxP1 expression, cells were bulk passaged by scraping and re-seeded in VTN-NC-coated chamber slides at a 1:200 ratio in E6 medium containing 1 μM RA and 100 nM purmorphamine (PM; Cayman Chemicals). RA/PM treatment was carried out for 7 days, followed by an additional 7 day treatment with 10 ng/ml brain-derived neurotrophic factor (BDNF; Peprotech), 10 ng/ml glial-derived neurotrophic factor (GDNF; Peprotech), and cAMP (1 μM; Sigma). For accelerated neuronal differentiation, neuromesodermal progenitors were treated with 1 μM RA, 2 μM PM, and 1 μg/ml recombinant sonic hedgehog (SHH) for 2 days, then passaged with ACCUTASE® and re-seeded in VTN-NC coated chamber slides at 1×10⁴ cells/cm² in E6 medium containing 1 μM RA, 1 μM DAPT (Cayman Chemicals), 10 ng/ml BDNF, 10 ng/ml GDNF, and 1 μM cAMP for 7 days.

Flow cytometry. Flow cytometry was conducted as described in the Examples above. Primary antibodies against Sox2 (mouse; 1:1000; Millipore), brachyury (goat; 1:200; R&D Systems), Pax6 (mouse; 1:200; DSHB), and HoxB4 (rat; 1:50; DSHB) were utilized. Samples were run on a FACSCaliber (BD Biosciences) and data were analyzed using Cyflogic software. Positive events were quantified by gating above the top 1% of species-matched IgG controls.

Immunocytochemistry. Immunocytochemistry was conducted as previously described. Primary antibodies against Sox2 (1:500), brachyury (1:500), Pax6 (rabbit; 1:500; Covance), N-cadherin (mouse; 1:500; BD Biosciences), Otx2 (goat; 1:500; R&D Systems), HoxB4 (rat; 1:50; DSHB), HoxDlO (goat; 1:500; R&D Systems), NeuN (mouse; 1:100; Millipore), Hb9 (mouse; 1:50; DSHB), ISL1 (mouse; 1:100; DSHB), and FoxP1 (rabbit; 1:20,000; Abcam) were utilized. Samples were visualized on a Nikon Ti-E epifluorescence microscope or a Nikon AIR confocal microscope. NIS-Elements software was used for image analysis.

qPCR. Total RNA was extracted from cultured cells using TRIZOL® (Life Technologies) according to the manufacturer's instructions. After isolation, 2-5 μg of total RNA was immediately subjected to reverse transcription using the Thermoscript™ RT-PCR kit (Life Technologies) in a 20 μL reaction according to the manufacturer's instructions. Resultant cDNA was diluted to 200 μL and utilized for qPCR (25 μL reactions, 1 μL cDNA per reaction) on a BioRad CFX96 detection unit using TAQMAN® Gene Expression Master Mix (Life Technologies) and TAQMAN® primers (Life Technologies). For all experiments, ΔΔCt values for each gene were calculated relative to ribosomal protein S18 (RPS18) RNA levels and converted to fold difference assuming 100% primer efficiency, then normalized to maximum expression levels as indicated in each figure legend.

Results. Recent animal studies have demonstrated that posterior neural tissue, which forms the hindbrain and spinal cord, is derived from bipotent neuromesodermal progenitors residing in the tail bud/stem zone/caudal lateral epiblast⁶⁻⁹. Stem zone progenitors exhibit colinear activation of HOX genes^{m}, which in turn prescribes neural progenitor positional identity along the posterior rostral/caudal axis and dictates neural circuit organization^{11, 12}. In vivo and ex vivo studies have demonstrated that Wnt/β-catenin and FGF signaling contribute to the induction of posterior identityl¹³⁻¹⁵ and FGF signaling maintains the stem-like phenotype of progenitors found in the caudal stem zone^{16, 17}. Some studies have suggested that temporal gradients of RA, FGF8, and GDF11 shape the rostral and caudal HOX profiles in hindbrain and spinal cord neuroectoderm^{12, 18-20,} but others have indicated that stem zone specification and HOX transcription requires FGF but not RA⁶. Overall, no clear consensus has been reached on the mechanisms that manipulate HOX expression patterns in posterior body formation.

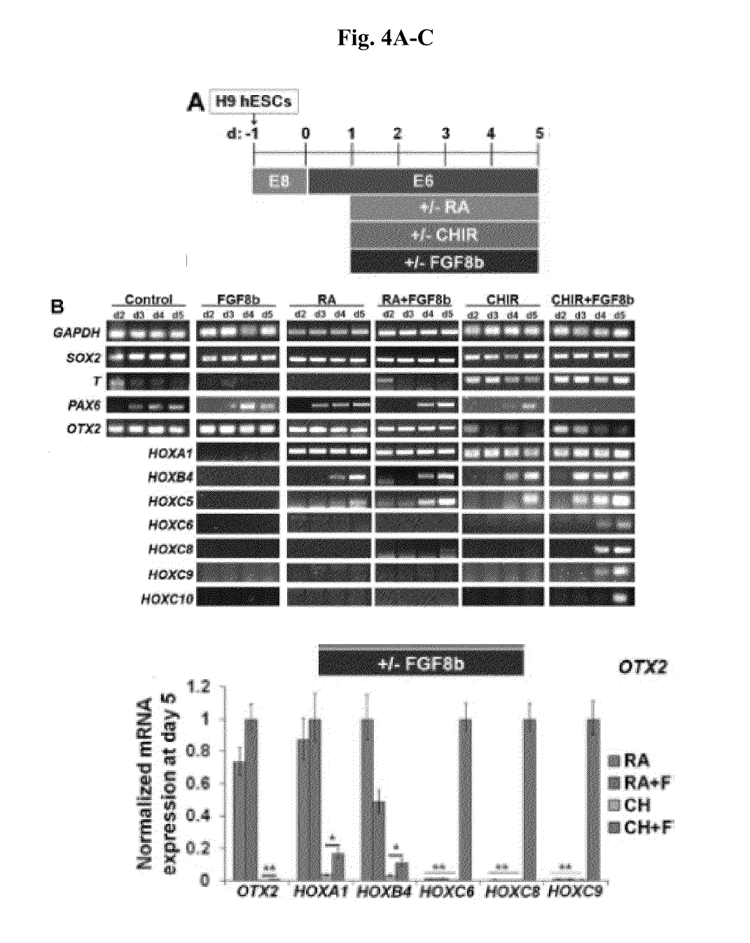

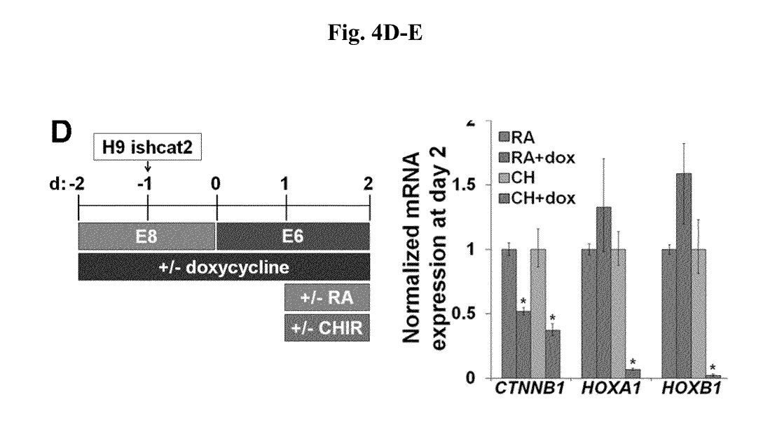

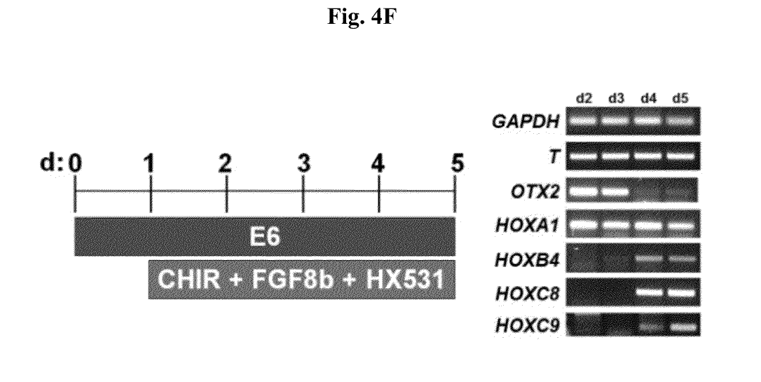

hPSC differentiation systems are an excellent resource to probe developmental cues in ways that are otherwise intractable in humans. Previous studies of neural differentiation from hPSCs have used RA to generate posterior neural progenitors, but these protocols generate cells mostly restricted to hindbrain and cervical spinal cord identity^{2, 3}. In an effort to determine the explicit extracellular cues required to generate a posterior neural phenotype in vitro, we first examined HOX expression by modifying a fully defined, monolayer system that differentiates hPSCs to highly pure neuroectoderm within 4 days²¹. We differentiated H9 hESCs²² in E6 medium for 24 h and then added RA, CHIR 99021 (CHIR; small molecule inhibitor of Gsk3 that stimulates canonical Wnt/β-catenin signaling²³), FGF8b (or selected other FGFs), or combinations of these morphogens and monitored HOX expression profiles by RT-PCR for 4 days (FIGS. 4A-C).

FGF8b alone did not induce HOX transcription, whereas either RA or CHIR 99021 induced progressive HOX activation from HOXA1 down to HOXC5 (FIG. 1B). More interestingly, the combination of RA and FGF8b did not alter HOX progression, but the combination of FGF8b and CHIR 99021 accelerated HOX progression to expression of paralogs found in the spinal cord such as HOXC6, HOXC8, and HOXC9, which exhibited >90-fold higher expression under the FGF8b/CHIR 99021 combination compared to the other morphogen treatments (FIGS. 4B-C). Moreover, adding HX531, an RXR inhibitor, did not prevent the ability of FGF8b/CHIR 99021 to activate cervical and thoracic HOX paralogs (FIG. 4F). In addition, CHIR 99021 quantitatively reduced OTX2 expression (associated with midbrain/forebrain identity) by >100-fold compared to RA, which also resulted in a widespread decrease in Otx2 protein levels (FIG. 4C and data not shown). These results indicate that both RA and Wnt/β-catenin can induce some degree of posterior identity judged by HOX1-5 paralog activation, which agrees with recent results from mouse ES cells¹⁹. However, RA by itself is insufficient to transition cells to an Otx2-posterior phenotype in the allotted experimental timeframe, whereas Wnt/β-catenin and FGF signaling cooperatively facilitate progression to cervical and thoracic identity.

To confirm the effects of CHIR 99021 were due to activation of β-catenin, we utilized the H9 ishcat2 line which harbors a doxycycline-inducible β-catenin shRNA cassette²³. Addition of doxycycline prior to CHIR 99021 treatment reduced CTNNB 1 (β-catenin) expression by 2.5-fold, which led to a decrease in HOXA1 expression by 15-fold and HOXB1 expression by 47-fold, indicating a functional role for β-catenin in initiating HOX transcription (FIG. 4D). Doxycycline also reduced CTNNB 1 by 2-fold in RA-treated cells, but did not reduce HOXA1 or HOXB1 expression, suggesting RA-mediated induction of HOX transcription is β-catenin-independent (FIG. 4D). We extended doxycycline with FGF8b/CHIR 99021 for 4 days and observed a reduction in HOXC6 by 9-fold, HOXC8 by 7-fold, and HOXC9 by 8-fold, which implicates β-catenin in HOX progression to caudal paralogs (FIG. 4E).

We next evaluated the necessity of Wnt/β-catenin and FGF signaling for establishing the neuromesodermal state (identified in vitro by co-expression of the mesoderm marker T/brachyury and neural marker Sox2) from which posterior neural tissue is derived⁸. Four days of combined CHIR 99021 and FGF8b treatment induced >100-fold more T expression compared to CHIR 99021 alone and >10,000-fold more expression than combined RA and FGF8b treatment (data not shown), and doxycycline treatment in the ishcat2 line reduced T by 30-fold (FIG. 4E), indicating a cooperative role for Wnt/β-catenin and FGF signaling in T maintenance. At the protein level, simultaneous treatment with FGF8b and CHIR 99021 induced uniform brachyury but caused a sharp decrease in Sox2 expression (23±0% Sox2+), indicating a fate shift exclusively towards mesoderm (FIG. 2a-b). Conversely, pre-treatment with FGF8b prior to CHIR 99021 yielded uniform expression of both brachyury and Sox2 (FIG. 2a-b).

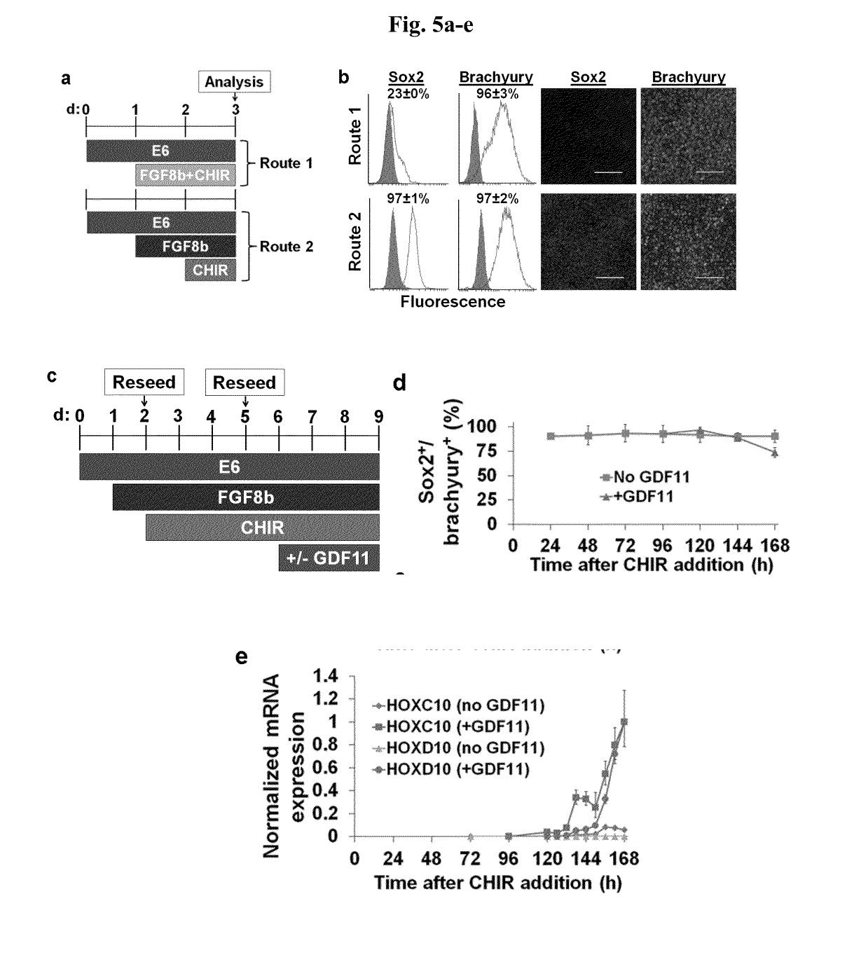

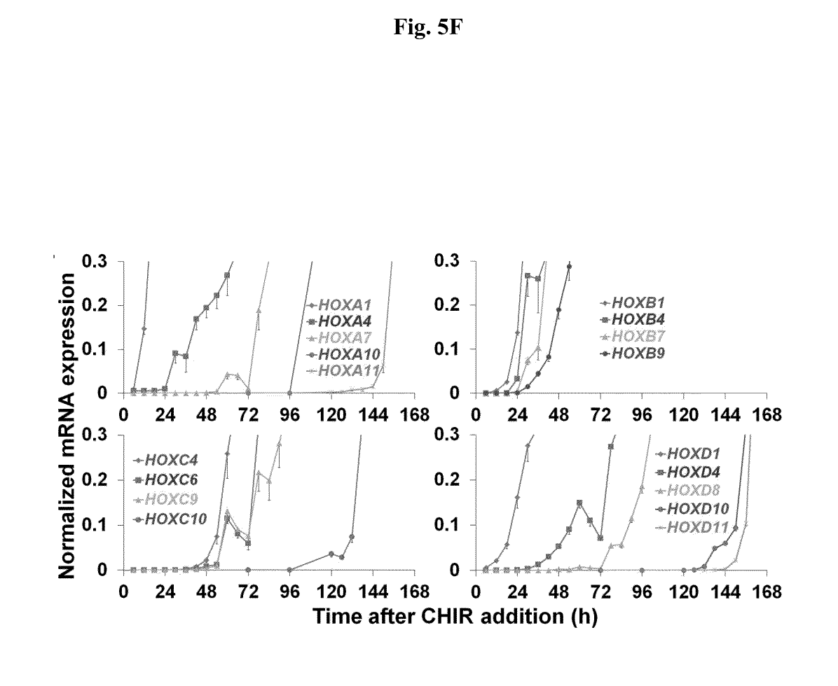

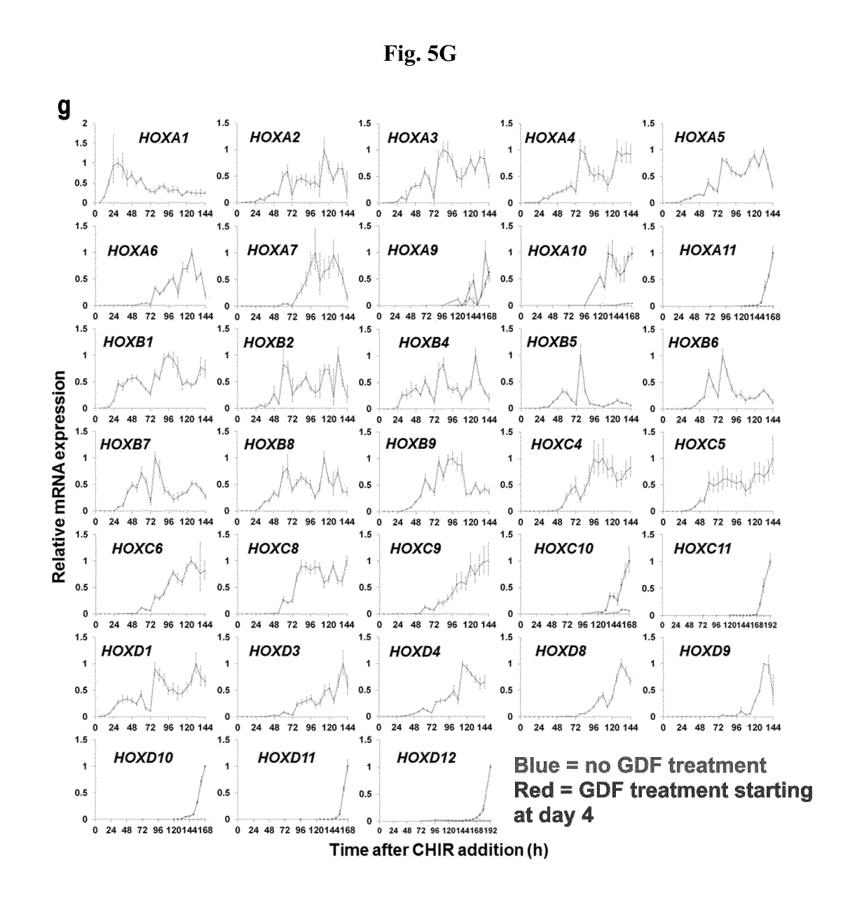

We could maintain highly pure neuromesodermal cultures (75-100% Sox2⁺/brachyury⁺) for 7 days that yielded colinear activation of the majority of HOX paralogs (FIGS. 5c-g). After activation, rostral HOX transcripts remain expressed even after activation of caudal paralogs and fluctuate within a ˜5-fold range (data not shown). Growth differentiation factor 11 (GDF11, a TGFβ family member expressed at later stages of stem zone progression^{18, 20}) was necessary for robust activation of lumbar (HOX10) and sacral (HOX11) paralogs²⁴ (FIG. 5e and g), but did not repress hindbrain/cervical genes HOXA4, HOXB4, and HOXC6 (data not shown), nor disrupt the neuromesodermal state (FIG. 5d).

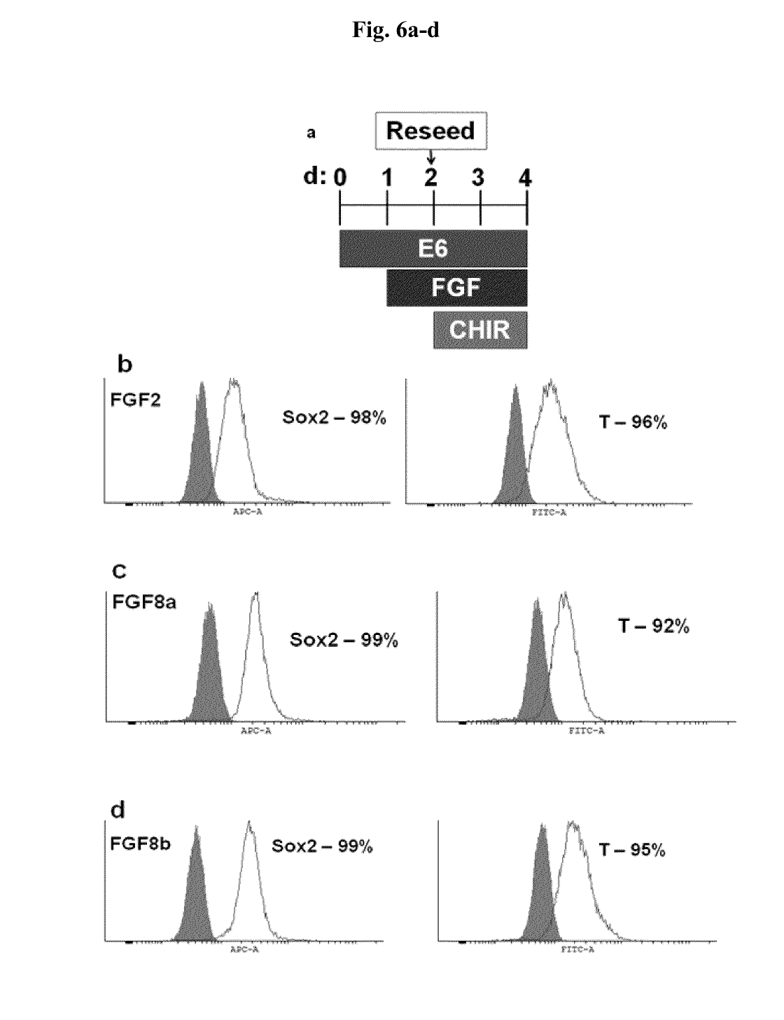

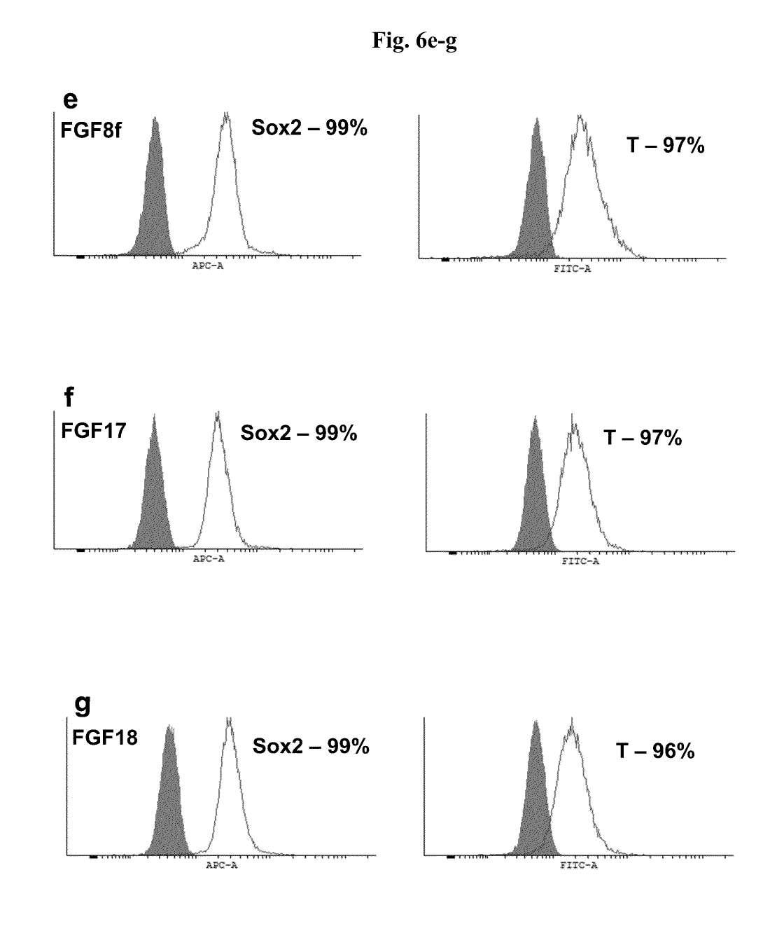

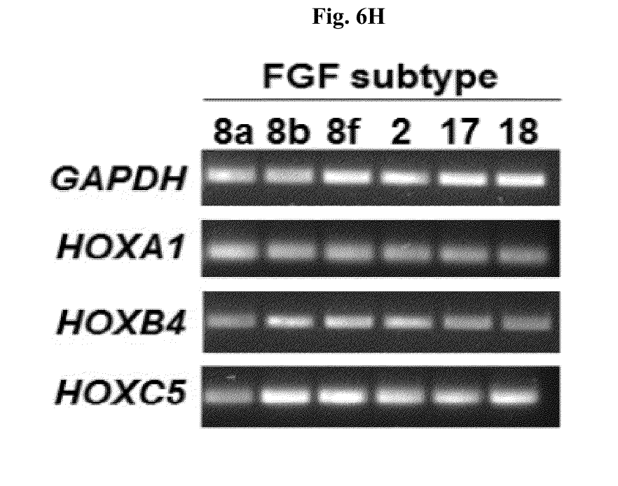

We further demonstrated that other FGFs, including FGF2, FGF8a, FGF8f, FGF17 and FGF18, could be successfully used in conjunction with CHIR 99021 in our protocol instead of FGF8b (FIGS. 6A-H). Specifically, FGF8a, 8b, and 8f, FGF17, FGF18, and FGF2 were used to execute our HOXpatterning protocol on concurrent neurally differentiating hPSC cultures. The ability of alternative FGFs to recapitulate the effects of FGF8b was assessed by their ability to maintain a Sox2⁺/T⁺ neuromesodermal state while also inducing a similar rate of sequential HOX gene expression. Post FGF/CHIR addition, flow cytometry assays for Sox2 and T were performed, and the rate of sequential HOX gene expression was assessed by RT-PCR for a select panel of HOX genes on collected mRNA samples.

As shown in FIG. 6b-g, all of the FGFs tested were capable of maintaining a Sox2⁺/Brachyury (T)⁺ neuromesodermal state. Hox1-5 paralogs were also activated in the cell populations within 2 days of FGF/CHIR treatment (FIG. 6H).

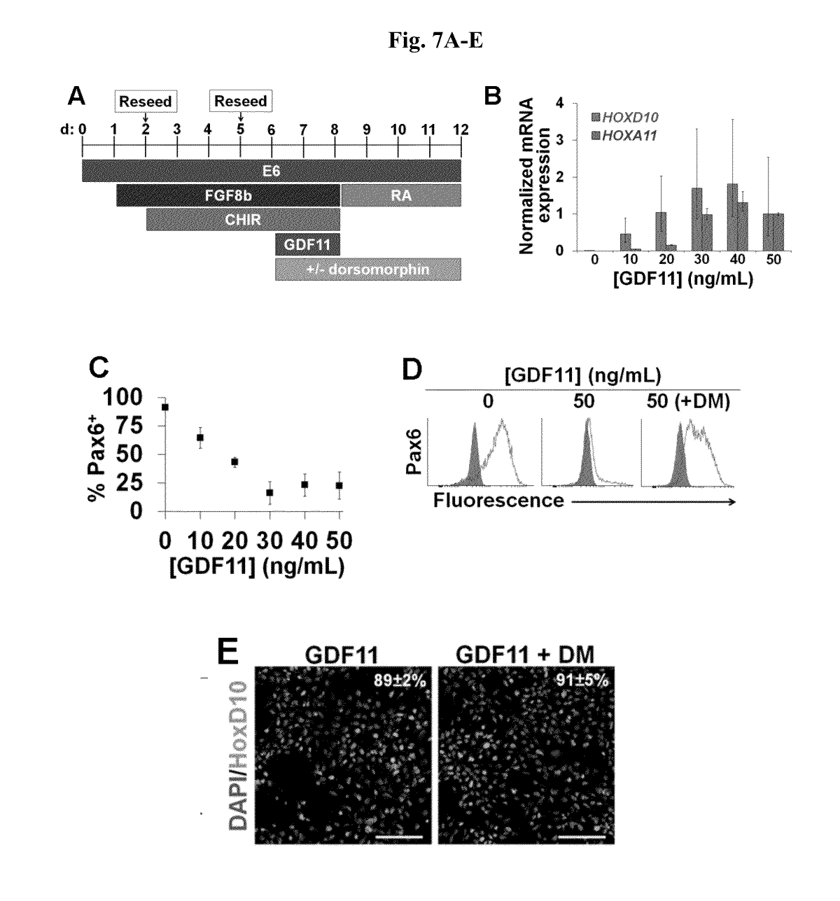

After neural tube closure in vivo, opposing gradients of TGFβ and SHH signaling produce distinct dorsal and ventral domains from which sensory neurons, interneurons, and motor neurons arise. Pax6, which has been identified as a neuroectoderm fate determinant, is expressed throughout most of the neural tube but is absent from the most dorsal and ventral domains. When RA is added to neuromesodermal cells at cervical and thoracic levels, Pax6 is uniformly induced, but when GDF11 has been present in the neuromesodermal morphogen cocktail, lumbar HOX genes are activated (FIG. 7B) but Pax6 expression is decreased (FIG. 7C). The ability of GDF11 to control rostral/caudal patterning is mediated by signaling via ALK5 and activation of the SMAD2/3 complex. GDF11 can also activate SMAD1/5/8 in vitro, and activation of this SMAD complex contributes to dorsal patterning. Since dorsomorphin selectively inhibits ALK2, ALK3, and ALK6, which blocks SMAD1/5/8 signaling, we hypothesized the addition of dorsomorphin should prevent acquisition of a dorsal phenotype without affecting rostral/caudal patterning. Indeed, the addition of dorsomorphin with GDF11 and throughout RA treatment was sufficient to recover Pax6 expression (83±4% Pax6⁺; FIG. 7D), and dorsomorphin treatment did not reduce HoxD 10 expression compared to cells receiving the standard FGF8b/CHIR 99021/GDF11 cocktail prior to RA treatment (FIG. 7E).

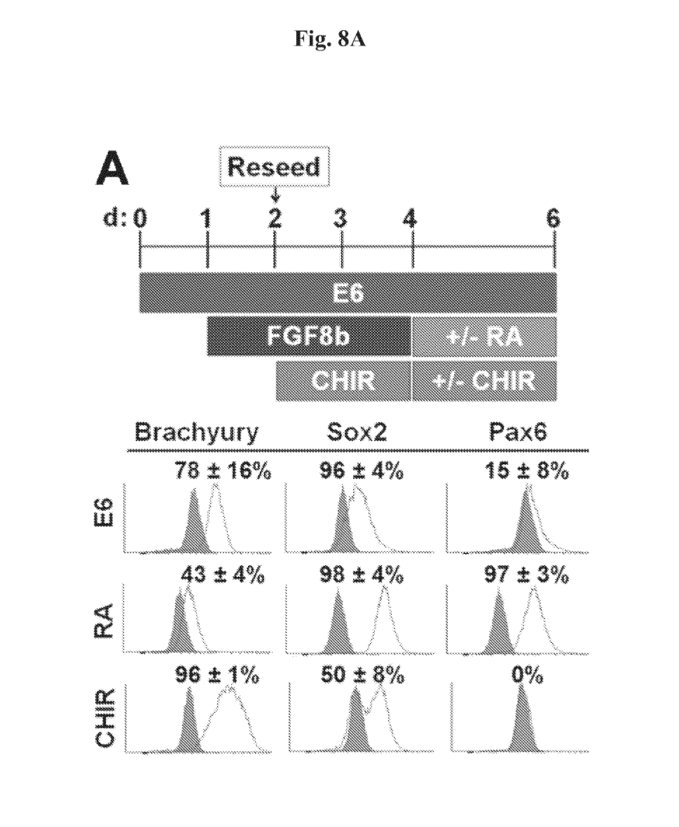

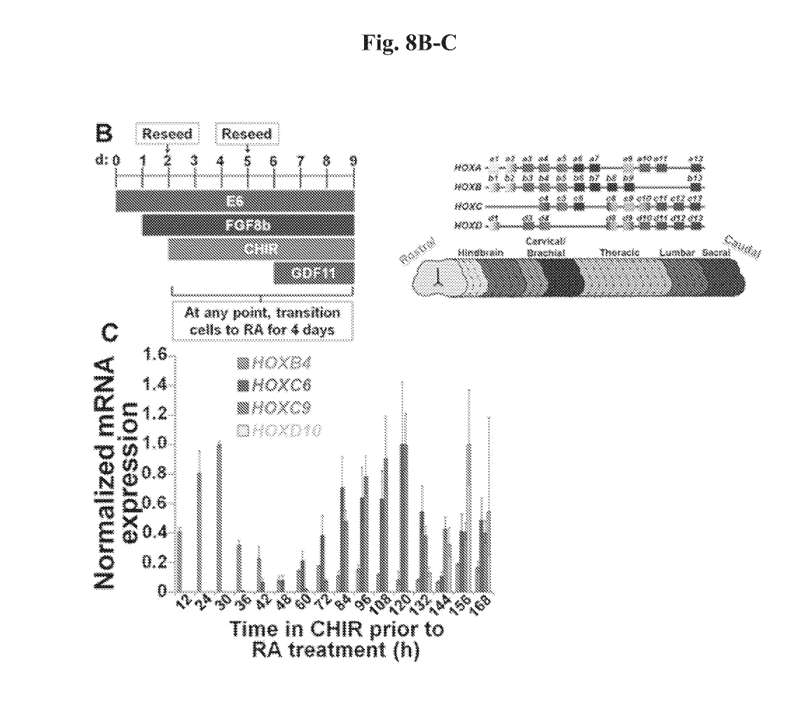

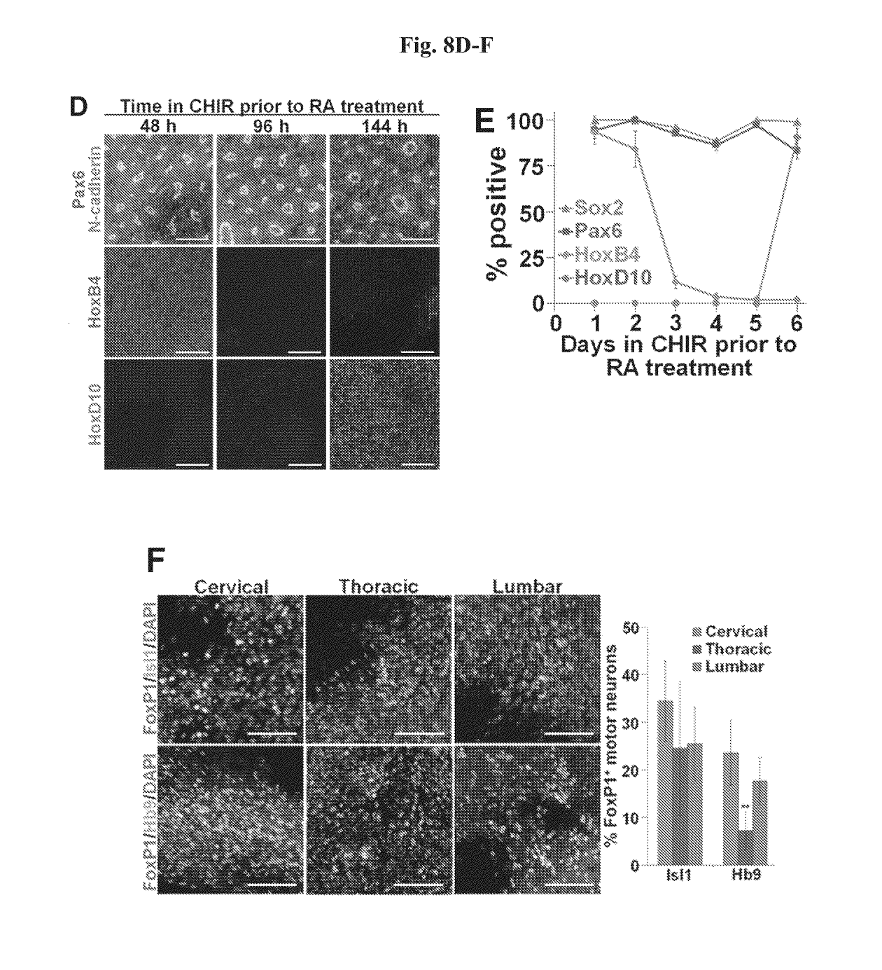

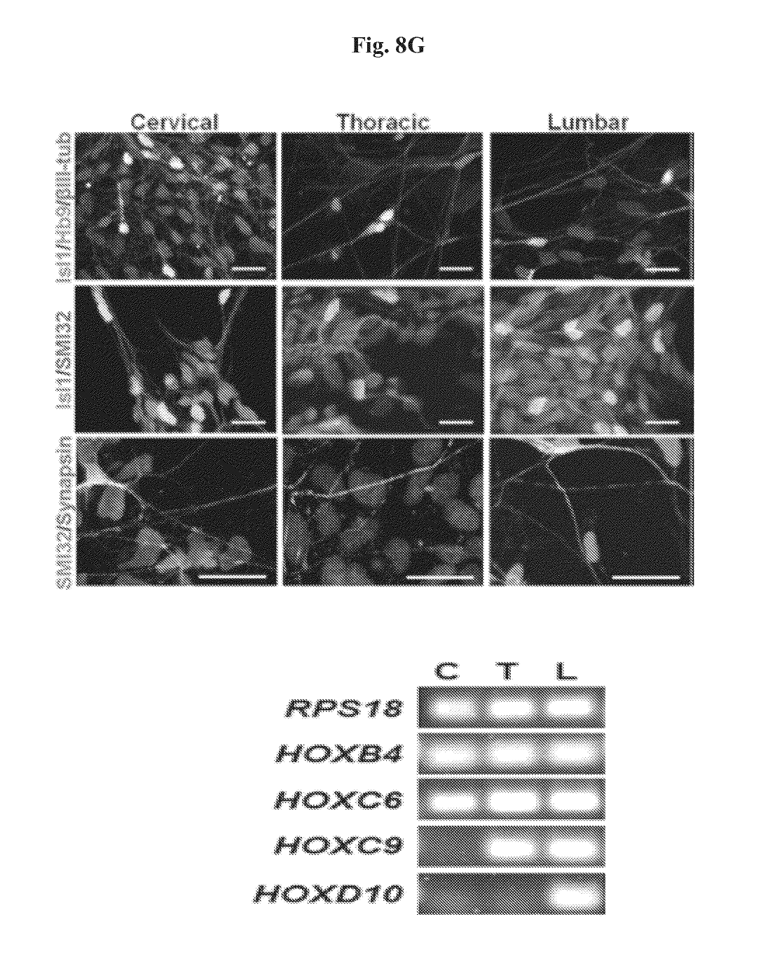

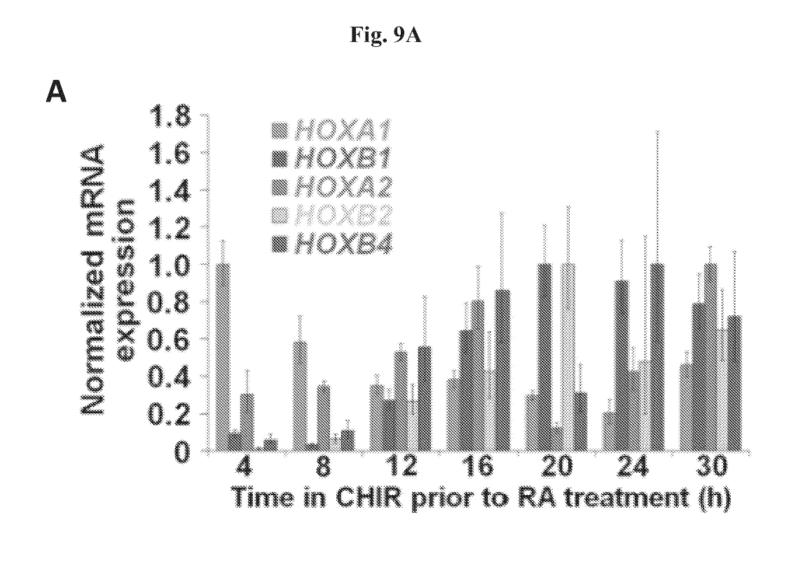

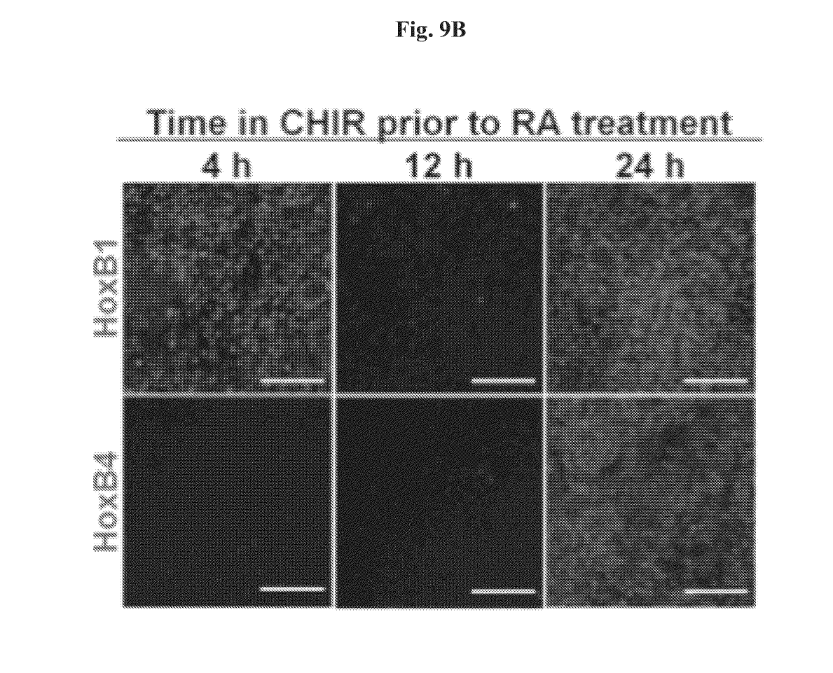

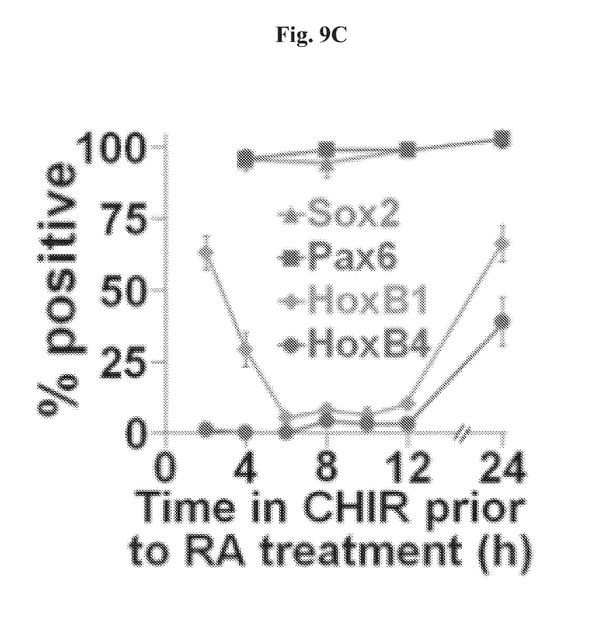

We next evaluated the transition of neuromesodermal progenitors to definitive neural progenitors. RA secreted from somites in response to declining FGF signaling elicits dorsal/ventral patterning and expression of the neuroectoderm marker Pax6 in vivo^{16, 26}, which led us to suspect RA serves as both the neuroectoderm fate switch and HOX ‘stop signal’. We first investigated the ability of RA to initiate neuroectoderm specification from neuromesodermal progenitors. As shown in FIG. 8A, neuromesodermal progenitors exposed to E6 medium alone maintained Sox2 expression but did not gain Pax6 expression (96±4% Sox2⁺, 15±8% Pax6⁺), while exposure to only CHIR induced a mesodermal fate shift as evidenced by Sox2 down-regulation, no Pax6 induction, and uniform maintenance of Brachyury expression (50±8% Sox2⁺, 96±1% Brachyury⁺). If RA was included in the E6 medium, Pax6 expression was uniformly induced with Sox2 (98±4% Sox2⁺, 97±3% Pax6⁺) and brachyury was decreased (43±4%), indicating commitment to the neuroectoderm lineage. Addition of CHIR 99021 or FGF8b with RA repressed its ability to induce Pax6 expression, and CHIR 99021 in the absence of FGF8b and RA reduced Sox2 while maintaining brachyury (96±1% brachyury⁺50±8% Sox2⁺), indicating a fate shift towards mesoderm. At any point in neuromesodermal differentiation, the addition of RA generated highly pure (>83%) Pax6⁺ neuroectoderm (FIG. 8B-E and FIG. 9A-C). Moreover, RA was sufficient to arrest HOX progression and yield HOX expression profiles commensurate to their status in the neuromesodermal state, and this fixed HOX state was maintained upon extended differentiation to motor neurons (FIG. 8G).

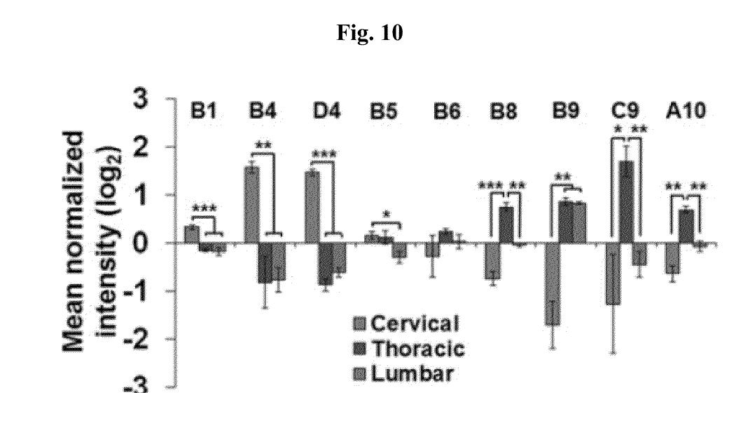

In the neuroectoderm state, cross-repressive interactions between Hox factors²⁷⁻²⁹ generated protein profiles dependent on which HOX transcripts were activated prior to RA treatment. HoxB4 was uniformly expressed if RA was added between 24-48 h of neuromesodermal propagation, but its expression dropped greatly within 24 h of HOXC9 induction (84±10% down to 12±4% HoxB4⁺ cells), whereas HoxC9 is known to directly repress rostral Hox paralogs. Similarly, HoxD10 was not detected until after GDF11 treatment, increasing from 0% to 89±2% when RA was added between 120 and 144 h of neuromesodermal propagation. See FIGS. 8B-E and FIG. 10.

We tested the ability of RA to generate NSC cultures with distinct and region-specific HOX expression profiles in accordance with the temporal progression of colinear transcriptional activation in the neuromesodermal state. We also assessed cross-repressive interactions between HOX transcription factors that would demarcate individual hindbrain and spinal cord domains.

Specifically, we first assessed hindbrain/cervical spinal cord cultures (0-30 h FGF8b/CHIR treatment prior to RA) (FIGS. 9A-C). qPCR analysis revealed elevated expression of HOXA1 and HOXA2 prior to HOXB1, HOXB2, and HOXB4, and analysis of protein expression demonstrated that early cultures were HoxB1+/HoxB4− (2 h FGF8b/CHIR, 4 d RA; 63±6% HoxB1+) but rapidly lost HoxB1 expression after only 4 additional h (6 h FGF8b/CHIR, 4 d RA; 5±2% HoxB1+) (FIG. 9A-C). By the 24 h mark, NSCs reacquired HoxB1 and now also expressed HoxB4 (63±5% HoxB1+; 37±8% HoxB4+) (FIG. 9A-C). Interestingly, the high percentage of HoxB1+ cells at the 2 and 4 h mark coincided with low HOXB1 expression (˜10% of the maximum observed expression at 20 h), suggesting that while transcript levels are useful for identifying relative HOX expression, they may not be instructive for predicting HOX expression and the establishment of rhombomeric and vertebral domains. These results also indicate that HOX cycling in the culture dish is synced within roughly 6 h.

We repeated the above set of experiments but next focused on later time points that would be analogous to the spinal cord, where caudal HOX factors typically repress more rostral paralogs to establish cervical, thoracic, and lumbar domains. Via immunocytochemistry and flow cytometry, we observed that HoxB4 was uniformly expressed in NSC cultures when RA was added between 24-48 h of neuromesoderm propagation, but its expression dropped sharply (84±10% down to 12±4% HoxB4+ cells) if RA was added 24 h later (FIG. 8C-E); this coincides with the initiation of thoracic HOXC9 expression, which is known to repress Hox4 paralog expression in vivo³² . Moreover, HoxD10 was only detected in NSC cultures when RA was added after ˜144 h of neuromesoderm propagation with GDF11 treatment (FIGS. 8C-E), and HoxB4 repression was maintained after HoxD10 induction (FIGS. 8D-E and FIG. 10). Similar to the hindbrain cultures detailed earlier, these results are highly indicative of region-specific patterning.

Additional insights into the diversity of HOX expression were obtained using quantitative mass spectrometry on NSC cultures patterned to cervical, thoracic, and lumbar spinal cord domains (FIG. 10). As expected, HoxC9 was expressed in thoracic NSCs but repressed in the HoxD10⁺ lumbar culture, and many (HoxB1, HoxB4, HoxD4, HoxB5, HoxB8, HoxC9, and HoxA10) but not all (HoxB9) detected HOX factors exhibited expression patterns indicative of similar cross-repressive interactions (FIG. 10). Similar to the hindbrain cultures, repression of Hox proteins occurred even though HOX transcripts remained expressed, possibly indicating cross-repressive interactions occur at the post-transcriptional stage. Collectively, these results provide strong evidence that finely-tuned rostrocaudal patterning can be produced in vitro using completely defined morphogen regimes.

Finally, we sought to differentiate cervical, thoracic, and lumbar neuromesodermal progenitors to Isl1⁺/Hb9⁺ motor neurons (FIG. 8F) exhibiting appropriate co-expression of accessory transcription factor FoxP1 that demarcates limb innervating columnar identity. Accelerated differentiation by treatment with the Notch signaling inhibitor DAPT yielded >95% NeuN⁺ neuronal lineage cells with widespread expression of βIII-tubulin within 7 days. Motor neurons were identified by co-expression of βIII-tubulin with Isl1 or Hb9, and co-expression of neurofilament H (SMI-32) and synapsin demonstrated motor neuron maturity (FIG. 8G). Relative HOX profiles from the neural progenitor state were retained, indicating positional identity is relatively unchanged after neuronal maturation.

Motor neurons reside in five distinct motor columns in vivo: the lateral motor column (LMC) located in the brachial (caudal cervical) and lumbar spinal cord, the preganglionic column (PGC) located in the thoracic spinal cord, the hypaxial motor column (HMC) located in the thoracic spinal cord, the phrenic motor column (PMC) located in the rostral cervical spinal cord, and the medial motor column (MMC) that extends throughout the cervical, thoracic, and lumbar spinal cord. Thus, at the brachial and lumbar levels, motor neurons reside in the LMC or MMC, and at thoracic levels, motor neurons reside in the PGC, HMC, or MMC. Consequently, columnar identity can be demarcated by combinatorial expression of specific transcription factors. MMC motor neurons exclusively express Lhx3, whereas LMC motor neurons express Lhx 1 and FoxP1 and PGC motor neurons express lower levels of FoxP19. The LMC can be further divided into medial and lateral domains, where the medial LMC contains only FoxP1⁺/Isl1⁺/Hb9⁻ motor neurons and the lateral LMC contains FoxP1+ motor neurons expressing Hb9 but not Isl111. Moreover, PGC motor neurons express Isl1 but not Hb9. Isl1 expression can also be found in some dorsal interneurons and dorsal root ganglion sensory neurons, but ventral induction by activation of sonic hedgehog (SHH) signaling should limit the generation of these cell types in vitro.

As expected, similar numbers of Isl1⁺/FoxP1⁻ motor neurons were derived from cervical, thoracic, and lumbar neuroectoderm (35±8%, 25±14%, and 25±8%, respectively, of the total Isl1⁺ population, FIG. 8F), whereas Hb9⁺/FoxP1⁺ motor neurons were primarily differentiated from cervical and lumbar neuroectoderm (24±7% and 18±5% of the total Hb9+ population) with significantly fewer numbers derived from thoracic neuroectoderm (7±4%). Thus, the FoxP1⁺ motor neuron population approximates the percentages described above, with the caveat that Isl1 and Hb9 may overlap to some degree in the cervical and lumbar regions. While it has been observed in vivo that PGC motor neurons express lower levels of FoxP1 than LMC motor neurons, we assumed any positive labeling of FoxP 1 in the thoracic samples, without consideration of high or low expression, was indicative of PGC identity due to inherent difficulties in comparing expression levels across samples that were fixed and stained at different times.

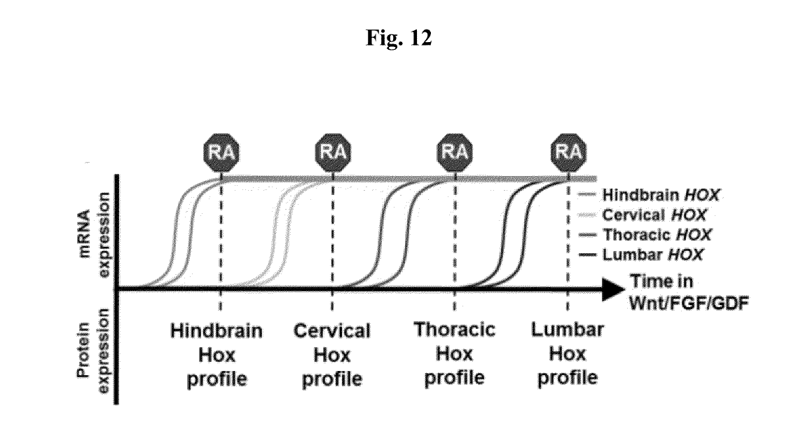

Overall, this method is the first example of deterministic HOX patterning in human cells which allows predictable generation of highly pure posterior neural progenitors possessing a fixed positional identity based on timed exposure to morphogens, as outlined in FIG. 12. The fully defined and scalable nature of this protocol will provide significant utility towards studying HOX regulatory networks in ways that were previously intractable in human systems. Moreover, as recent work has suggested, positional identity of hPSC-derived neural cells is crucial for their therapeutic efficacy^{30, 31}, and because HOX expression patterns are crucial determinants of cellular organization and integration in the developing spinal cord^{12, 27, 28, 32,} the methods described herein lay the groundwork for future advancements in disease modeling and regenerative therapies.

### REFERENCES FOR EXAMPLE 4

1. Mallo, M., Wellik, D. M. & Deschamps, J. Hox genes and regional patterning of the vertebrate body plan. Dev Biol 344, 7-15 (2010).
2. Li, X. J. et al. Specification of motoneurons from human embryonic stem cells. Nat Biotechnol 23, 215-221 (2005).
3. Amoroso, M. W. et al. Accelerated high-yield generation of limb-innervating motor neurons from human stem cells. Journal of Neuroscience 33, 574-586 (2013).
4. Lengerke, C. et al. Hematopoietic development from human induced pluripotent stem cells. Ann N Y Acad Sci 1176, 219-227 (2009).
5. Patani, R. et al. Retinoid-independent motor neurogenesis from human embryonic stem cells reveals a medial columnar ground state. Nat Commun 2, 214 (2011).
6. Delfino-Machin, M., Lunn, J. S., Breitkreuz, D. N., Akai, J. & Storey, K. G. Specification and maintenance of the spinal cord stem zone. Development 132, 4273-4283 (2005).
7. Kondoh, H. & Takemoto, T. Axial stem cells deriving both posterior neural and mesodermal tissues during gastrulation. Curr Opin Genet Dev 22, 374-380 (2012).
8. Takemoto, T. et al. Tbx6-dependent Sox2 regulation determines neural or mesodermal fate in axial stem cells. Nature 470, 394-398 (2011).
9. Tzouanacou, E., Wegener, A., Wymeersch, F. J., Wilson, V. & Nicolas, J. F. Redefining the progression of lineage segregations during mammalian embryogenesis by clonal analysis. Dev Cell 17, 365-376 (2009).
10. Soshnikova, N. & Duboule, D. Epigenetic temporal control of mouse Hox genes in vivo. Science 324, 1320-1323 (2009).
11. Narita, Y. & Rijli, F. M. Hox genes in neural patterning and circuit formation in the mouse hindbrain. Curr Top Dev Biol 88, 139-167 (2009).
12. Philippidou, P. & Dasen, J. S. Hox genes: choreographers in neural development, architects of circuit organization. Neuron 80, 12-34 (2013).
13. Nordstrom, U., Jessell, T. M. & Edlund, T. Progressive induction of caudal neural character by graded Wnt signaling. Nat Neurosci 5, 525-532 (2002).
14. Nordstrom, U., Maier, E., Jessell, T. M. & Edlund, T. An early role for WNT signaling in specifying neural patterns of Cdx and Hox gene expression and motor neuron subtype identity. PLoS Biol 4, e252 (2006).
15. Takemoto, T., Uchikawa, M., Kamachi, Y. & Kondoh, H. Convergence of Wnt and FGF signals in the genesis of posterior neural plate through activation of the Sox2 enhancer N-1. Development 133, 297-306 (2006).
16. Bertrand, N., Medevielle, F. & Pituello, F. FGF signalling controls the timing of Pax6 activation in the neural tube. Development 127, 4837-4843 (2000).
17. Mathis, L., Kulesa, P. M. & Fraser, S. E. FGF receptor signalling is required to maintain neural progenitors during Hensen's node progression. Nat Cell Biol 3, 559-566 (2001).
18. Liu, J. P., Laufer, E. & Jessell, T. M. Assigning the positional identity of spinal motor neurons: rostrocaudal patterning of Hox-c expression by FGFs, Gdfll, and retinoids. Neuron 32, 997-1012 (2001).
19. Mazzoni, E. O. et al. Saltatory remodeling of Hox chromatin in response to rostrocaudal patterning signals. Nat Neurosci 16, 1191-1198 (2013).
20. Liu, J. P. The function of growth/differentiation factor 11 (Gdfl1) in rostrocaudal patterning of the developing spinal cord. Development 133, 2865-2874 (2006).
21. Lippmann, E. S., Estevez-Silva, M. C. & Ashton, R. S. Defined human pluripotent stem cell culture enables highly efficient neuroepithelium derivation without small molecule inhibitors. Stem Cells (2013).
22. Thomson, J. A. et al. Embryonic stem cell lines derived from human blastocysts. Science 282, 1145-1147 (1998).
23. Lian, X. et al. Robust cardiomyocyte differentiation from human pluripotent stem cells via temporal modulation of canonical Wnt signaling. Proc Natl Acad Sci US A 109, E1848-1857 (2012).
24. Wellik, D. M. & Capecchi, M. R. Hox10 and Hox11 genes are required to globally pattern the mammalian skeleton. Science 301, 363-367 (2003).
25. Yu, J. et al. Induced pluripotent stem cell lines derived from human somatic cells. Science 318, 1917-1920 (2007).
26. Diez del Corral, R. et al. Opposing FGF and retinoid pathways control ventral neural pattern, neuronal differentiation, and segmentation during body axis extension. Neuron 40, 65-79 (2003).
27. Dasen, J. S., Liu, J. P. & Jessell, T. M. Motor neuron columnar fate imposed by sequential phases of Hox-c activity. Nature 425, 926-933 (2003).
28. Dasen, J. S., Tice, B. C., Brenner-Morton, S. & Jessell, T. M. A Hox regulatory network establishes motor neuron pool identity and target-muscle connectivity. Cell 123, 477-491 (2005).
29. Jung, H. et al. Global control of motor neuron topography mediated by the repressive actions of a single hox gene. Neuron 67, 781-796 (2010).
30. Ma, L. et al. Human embryonic stem cell-derived GABA neurons correct locomotion deficits in quinolinic acid-lesioned mice. Cell Stem Cell 10, 455-464 (2012).
31. Kriks, S. et al. Dopamine neurons derived from human ES cells efficiently engraft in animal models of Parkinson's disease. Nature 480, 547-551 (2011).
32. Dasen, J. S. & Jessell, T. M. Hox networks and the origins of motor neuron diversity. Curr Top Dev Biol 88, 169-200 (2009)

### Example 5

### Successful Patterning of iPSCs

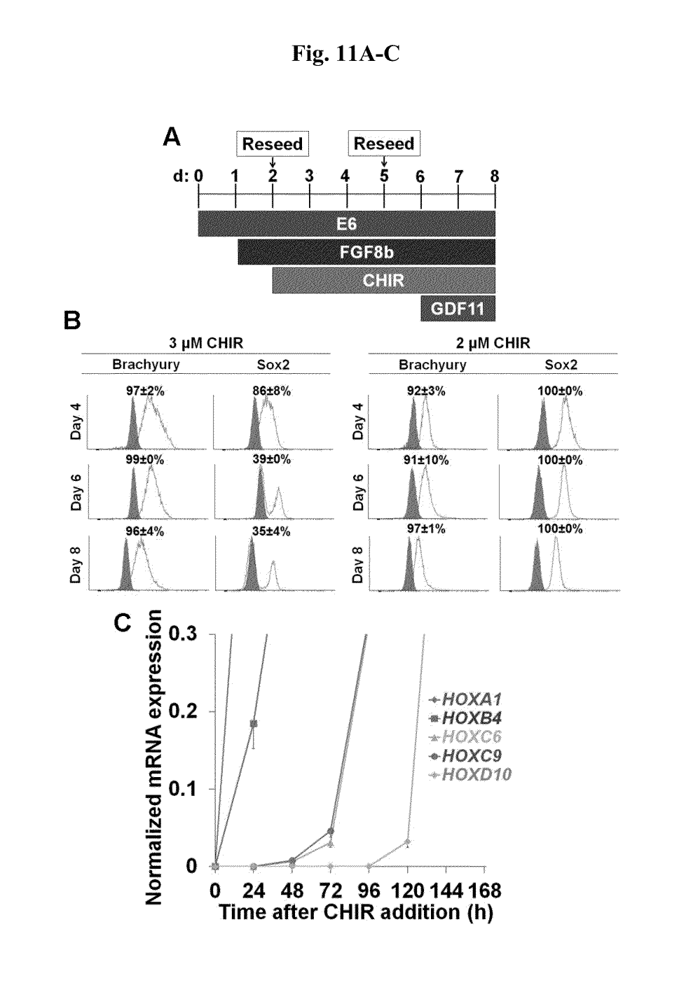

In this Example, we demonstrate that the patterning method disclosed in the previous examples using human embryonic stem cells can also be used with iPSCs. Specifically, we demonstrated neuromesodermal differentiation from IMR90-4 iPSCs by evaluating neuromesodermal propagation and HOX expression. Differentiation was conducted according to the protocol of FIG. 11A. Neuromesodermal identity was assessed during differentiation in both 2 and 3 μM CHIR (FIG. 11B). Grey histograms are IgG control, and red histograms are the label of interest. Whereas 2 μM CHIR maintained the neuromesodermal state, 3 μM CHIR resulted in a mesodermal shift, exemplified by a reduction in Sox2 expression (FIG. 11B). During neuromesodermal propagation, progressive HOX activation was verified by qPCR (FIG. 11C). The results demonstrate that, similar to the results obtained using hESCs, HOX activation was observed in neuromesoderm derived from IMR90-4 iPSCs.

### Example 6

### Effects of Signaling Pathway Inhibitors on FGF's Ability to Maintain Neuromesodermal Identity

In performing the protocols outlined in the Examples above, FGF maintains a pro-neural (Sox2⁺) state and prevents the culture from becoming mesenchymal (Sox2⁻/T⁺) due to the signaling effects of CHIR. In an attempt to identify which downstream signaling pathways are responsible for FGF's effects, we performed our protocol with various combinations of a PI3K inhibitor (LY-294002), an MEK inhibitor (U0126), and a PKC inhibitor (GF109203X), and measured Sox2 and T levels using flow cytometry.

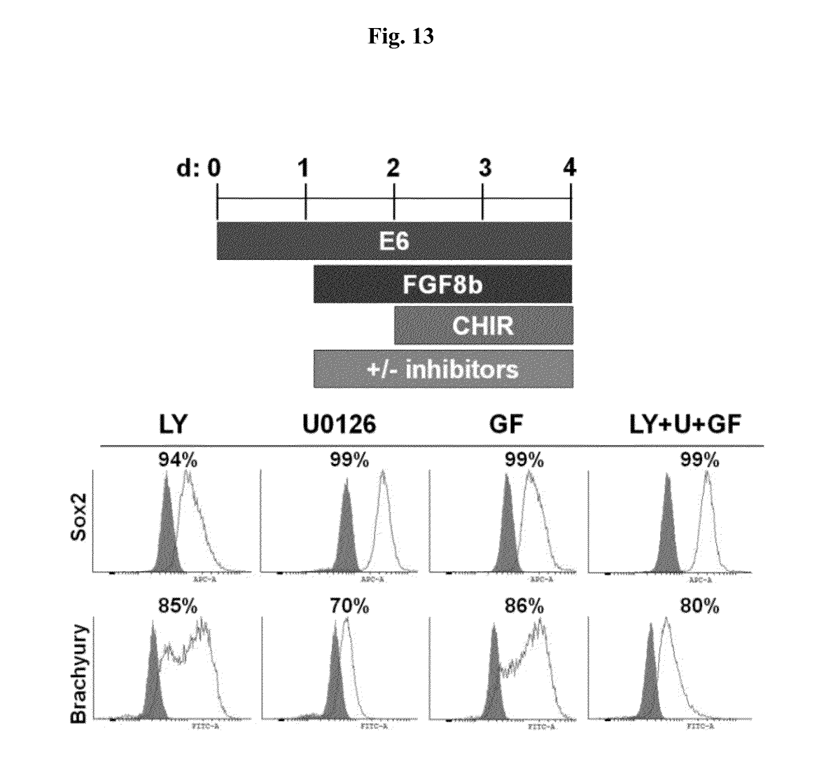

Specifically, we added LY-294002 (10 μM), U0126 (10 μM), GF109203X (3 μM, i.e. maximum dose that did not kill all cells), or a combination of all three inhibitors at 1 μM, and assayed for Sox2 and Brachyury (T) expression after 2 days. All cultures were 99% Sox2+ and 70-85% Brachyury+, indicating no shift towards an exclusive mesoderm identity (FIG. 13). Thus, pathways other than the ones listed above may be responsible for FGF's ability to maintain neuromesodermal identity. Furthermore, no inhibitor combination inhibited early stages of colinear HOX activation (data not shown), which is consistent with our findings that FGF signaling alone does not activate HOX genes (FIG. 4B).

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

What is claimed is:

1. A fully defined cell culture medium comprising water, salts, amino acids, vitamins, a carbon source, a buffering agent, selenium, insulin, an FGF, GDF11, and an activator of β-catenin pathway signaling, wherein the medium is substantially free of a TGFβ-signaling activator, and wherein the FGF is FGF2, FGF8a, FGF8b, FGF8f, FGF17, or FGF18.

2. The fully defined cell culture medium of claim 1, further comprising one or more of the group consisting of an inhibitor of BMP signaling, ascorbate, and a transferrin.

3. The fully defined medium of claim 1, wherein the fully defined medium is substantially free of a TGFβ-signal inhibitor.

4. The fully-defined medium of claim 1, wherein the activator of β-catenin pathway signaling is a GSK3 kinase inhibitor.

5. The fully defined medium of claim 4, wherein the GSK3 kinase inhibitor is CHIR99021.

6. A cell culture comprising human Sox2⁺/Brachyury⁻/PAX6⁻/Otx2⁻ caudal lateral epiblasts and the fully-defined medium of claim 1.

7. A kit comprising an FGF, an activator of β-catenin pathway signaling, and one or both of GDF 11 and a retinoid.

8. The kit of claim 7, further comprising a cell culture medium comprising water, salts, amino acids, vitamins, a carbon source, a buffering agent, selenium, and insulin.

9. The kit of claim 7, further comprising an inhibitor of BMP signaling.

10. The kit of claim 7, wherein the FGF is FGF8b, FGF2, FGF8a, FGF17, or FGF18.

11. A method for generating caudal lateral epiblasts from human pluripotent stem cells, comprising:
(i) culturing human pluripotent stem cells during a first culture period of about one to two days with a neural differentiation base medium to obtain a first cell population;
(ii) culturing the first cell population for a second culture period of about one day to about four days in neural differentiation base medium supplemented with an FGF to obtain a second cell population, wherein the second cell population is Sox2⁺, Otx2⁺, Brachyury⁻ and Pax6⁻; and
(iii) culturing the second cell population for a third culture period of about one day to about seven days in neural differentiation base medium supplemented with an FGF and an activator of β-catenin pathway signaling to obtain caudal lateral epiblasts that are Sox2⁺, Brachyury⁺, Pax6⁻ and Otx2⁻, wherein the neural differentiation base medium comprises water, salts, amino acids, vitamins, a carbon source, a buffering agent, selenium, and insulin, and wherein the FGF is FGF2, FGF8a, FGF8b, FGF8f, FGF17, or FGF18.

12. The method of claim 11, wherein the activator of β-catenin pathway signaling is a GSK3 kinase inhibitor.

13. The method of claim 11, wherein, in step (iii) the third culture period is at least six days long, and wherein, after at least four days of the third culture period, the culture medium further comprises GDF 11 and an inhibitor of BMP signaling.

14. The method of claim 13, wherein the inhibitor of BMP signaling is dorsomorphin, noggin, DMH1, or LDN193189.

15. A method for generating posterior neuroectoderm or neuroepithelium from human pluripotent stem cells comprising culturing the Sox2⁺/Brachyury⁺/PAX6⁻/Otx2⁻ caudal lateral epiblasts obtained by the method of claim 11 during a fourth culture period in a neural differentiation base medium supplemented with a retinoid, wherein the fourth culture period lasts for about one to five days.

16. The method of claim 15, wherein the fourth culture period lasts for about four days.

17. The method of claim 15, wherein the neural differentiation base medium supplemented with a retinoid further comprises an inhibitor of BMP signaling.

18. The method of claim 15, wherein the human pluripotent stem cells are cultured as a monolayer during the first cell culture period.

19. A method for generating human posterior neuroectoderm or neuroepithelium, the method comprising culturing a population of Sox2⁺/Brachyury⁺/PAX6⁻/Otx2⁻ human caudal lateral epiblasts that express at least one HOX gene in a neural differentiation base medium supplemented with a retinoid to obtain posterior neuroectoderm or neuroepithelium that comprises Sox2⁻/PAX6⁺/Brachyury⁻ cells that express the at least one HOX gene, wherein the neural differentiation base medium comprises water, salts, amino acids, vitamins, a carbon source, a buffering agent, selenium, and insulin.

20. The method of claim 19, wherein the population of Sox2⁻/Brachyury⁺/PAX6⁻ human caudal lateral epiblasts are cultured for a period of about one to five days to obtain the posterior neuroectoderm or neuroepithelium.

21. The method of claim 20, wherein the population of Sox2⁻/Brachyury⁺/PAX6⁻ human caudal lateral epiblastsare cultured for a period of about four days to obtain the posterior neuroectoderm or neuroepithelium.

22. The method of claim 19, wherein the human caudal lateral epiblasts express one or more of the group consisting of Hoxd10, Hoxc9, Hoxb4, Hoxc6, and Hoxa2.

23. The method of claim 19, wherein the supplemented neural differentiation base medium further comprises an inhibitor of BMP signaling.

24. A method for generating a population of human motor neurons having a specified HOX gene expression profile, comprising culturing human posterior neuroectoderm or neuroepithelium having a specified HOX gene expression profile in a neural differentiation base medium supplemented with a retinoid and an activator of the Hedgehog signaling pathway to obtain the population of human motor neurons.

25. The method of claim 24, wherein the population of motor neurons obtained has a higher level of Hoxd10 mRNA than Hoxc9 mRNA.

26. The method of claim 24, wherein the culturing is for about 7 to 14 days.

27. An isolated cell population consisting essentially of one of:
(i) human caudal lateral epiblasts;
(ii) human neuroectodermal cells;
(iii) human neuroepithelial cells; and
(iv) human motor neurons;
wherein the isolated cell population has an mRNA expression profile characterized by a level of a first specified HOX gene mRNA higher than that of a second specified HOX gene mRNA, wherein the first specified HOX gene is Hoxa2 and the second specified HOX gene is Hoxb4; the first specified HOX gene is Hoxb4 and the second specified HOX gene is Hoxc6; the first specified HOX gene is Hoxc6 and the second HOX gene is Hoxb4; the first specified HOX gene is Hoxc6 and the second specified HOX gene is Hoxc9; the first specified HOX gene is Hoxc9 and the second specified HOX gene is Hoxc6; the first specified HOX gene is Hoxc9 and the second specified HOX gene is Hoxd10; or the first specified HOX gene is Hoxd10 and the second specified HOX gene is Hoxc9.

28. The isolated cell population of claim 27, wherein a plurality of the human caudal lateral epiblasts, neuroectodermal cells, or neuroepithelial cells are genetically modified.

29. The isolated population of claim 28, wherein the plurality of genetically modified human caudal lateral epiblasts, neuroectodermal cells, or neuroepithelial cells comprise an expression cassette or exogenous RNA encoding a fluorescent reporter protein, a growth factor, an extracellular matrix protein, or an antibody.

30. A cell culture comprising the isolated population of claim 28, and a neural differentiation base medium supplemented with:
(i) an FGF and an activator of β-catenin pathway signaling;
(ii) a retinoid; or
(iii) a retinoid and an inhibitor of BMP signaling;
wherein the neural differentiation base medium comprises water, salts, amino acids, vitamins, a carbon source, a buffering agent, selenium, and insulin, and wherein the FGF is FGF2, FGF8a, FGF8b, FGF8f, FGF17, or FGF18.

31. The cell culture of claim 30, wherein group (i) further comprises GDF11.


SEQUENCE LISTING (24 sequences)

SEQ ID NO: 1 - DNA, 20 bp, Artificial Sequence
  Forward primer sequence.
caccgtcaaggctgagaacg

SEQ ID NO: 2 - DNA, 20 bp, Artificial Sequence
  Reverse primer sequence.
gccccacttgattttggagg

SEQ ID NO: 3 - DNA, 19 bp, Artificial Sequence
  Forward primer sequence.
tggacagttacgcgcacat

SEQ ID NO: 4 - DNA, 21 bp, Artificial Sequence
  Reverse primer sequence.
cgagtaggacatgctgtaggt

SEQ ID NO: 5 - DNA, 20 bp, Artificial Sequence
  Forward primer sequence.
ggcaacctacgcaagatggc

SEQ ID NO: 6 - DNA, 20 bp, Artificial Sequence
  Reverse primer sequence.
tgagggctgtgtctgttcgg

SEQ ID NO: 7 - DNA, 20 bp, Artificial Sequence
  Forward primer sequence.
cttccctgagacccagttca

SEQ ID NO: 8 - DNA, 20 bp, Artificial Sequence
  Reverse primer sequence.
cagggttgggtacctgtcac

SEQ ID NO: 9 - DNA, 21 bp, Artificial Sequence
  Forward primer sequence.
ccagacatcttcatgcgagag

SEQ ID NO: 10 - DNA, 21 bp, Artificial Sequence
  Reverse primer sequence.
ggcaggtctcactttgttttg

SEQ ID NO: 11 - DNA, 20 bp, Artificial Sequence
  Forward primer sequence.
aaatcaggaagcagacccac

SEQ ID NO: 12 - DNA, 22 bp, Artificial Sequence
  Reverse primer sequence.
gtagccgtactctccaactttc

SEQ ID NO: 13 - DNA, 22 bp, Artificial Sequence
  Forward primer sequence.
tacccctggatgcgcaaagttc

SEQ ID NO: 14 - DNA, 20 bp, Artificial Sequence
  Reverse primer sequence.
tggtgttgggcaacttgtgg

SEQ ID NO: 15 - DNA, 21 bp, Artificial Sequence
  Forward primer sequence.
acagatttacccgtggatgac

SEQ ID NO: 16 - DNA, 21 bp, Artificial Sequence
  Reverse primer sequence.
agtgaggtagcggttaaagtg

SEQ ID NO: 17 - DNA, 23 bp, Artificial Sequence
  Forward primer sequence.
gaatgagggaagacgagaaagag

SEQ ID NO: 18 - DNA, 20 bp, Artificial Sequence
  Reverse primer sequence.
cataggcggtggaattgagg

SEQ ID NO: 19 - DNA, 21 bp, Artificial Sequence
  Forward primer sequence.
tttatggggctcagcaagagg

SEQ ID NO: 20 - DNA, 22 bp, Artificial Sequence
  Reverse primer sequence.
tccacttcatccttcggttctg

SEQ ID NO: 21 - DNA, 19 bp, Artificial Sequence
  Forward primer sequence.
agcacaaagaggagaaggc

SEQ ID NO: 22 - DNA, 20 bp, Artificial Sequence
  Reverse primer sequence.
cgtctggtacttggtgtagg

SEQ ID NO: 23 - DNA, 18 bp, Artificial Sequence
  Forward primer sequence.
aaaggagagggccaaagc

SEQ ID NO: 24 - DNA, 22 bp, Artificial Sequence
  Reverse primer sequence.
gcgtctggtgtttagtataggg